(19) United States
(12) Patent Application Publication
(10) Pub. No.: US 2022/0381930 A1
(43) Pub. Date: Dec. 1, 2022

(54) RADIATION DETECTOR, RADIOGRAPHIC IMAGING APPARATUS, AND METHOD OF MANUFACTURING RADIATION DETECTOR

(71) Applicant: FUJIFILM CORPORATION, Tokyo (JP)

(72) Inventors: Shinichi USHIKURA, Kanagawa (JP); Munetaka KATO, Kanagawa (JP); Haruyasu NAKATSUGAWA, Kanagawa (JP)

(21) Appl. No.: 17/816,992

(22) Filed: Aug. 2, 2022

Related U.S. Application Data
(63) Continuation of application No. PCT/JP2021/005105, filed on Feb. 10, 2021.

(30) Foreign Application Priority Data
Feb. 20, 2020 (JP) 2020-027529

(51) Int. Cl. G01T 1/20; A61B 6/00

(52) U.S. Cl. CPC G01T 1/2018; A61B 6/4208

(57) ABSTRACT

A radiation detector includes a sensor substrate, a conversion layer, and a reinforcing member. In the sensor substrate, a plurality of pixels that accumulate electric charges generated in response to light converted from radiation are formed in a pixel region of a first surface of a flexible base material, and the first surface is provided with a terminal for electrically connecting the flexible cable. The conversion layer is provided on the first surface of the base material 11 and converts the radiation into the light. The reinforcing member is provided in a region including at least a facing region, facing the terminal, on a second surface of the base material opposite to the first surface and has super engineering plastic as a material.

### CROSS-REFERENCE TO RELATED APPLICATION

This application is a continuation application of International Application No. PCT/JP2021/005105, filed Feb. 10, 2021, the disclosure of which is incorporated herein by reference in its entirety. Further, this application claims priority from Japanese Patent Application No. 2020-027529 filed on Feb. 20, 2020, the disclosure of which is incorporated herein by reference in its entirety.

### BACKGROUND

### 1. Technical Field

The present invention relates to a radiation detector, a radiographic imaging apparatus, and a method of manufacturing the radiation detector.

### 2. Description of the Related Art

In the related art, radiographic imaging apparatuses that perform radiographic imaging for medical diagnosis have been known. A radiation detector for detecting radiation transmitted through a subject and generating a radiographic image is used for such radiographic imaging apparatuses.

As this type of radiation detector, there is one comprising a conversion layer, such as a scintillator, which converts radiation into light, and a substrate in which a plurality of pixels, which accumulate electric charges generated in response to light converted in the conversion layer, are provided in a pixel region of a base material. A flexible base material is known as the base material of the substrate of such a radiation detector, and a cable used for reading out the electric charges accumulated in the pixels is connected to a terminal provided on the flexible base material. By using the flexible base material, for example, there is a case in which the weight of the radiographic imaging apparatuses (the radiation detectors) can be reduced, and a subject is easily imaged.

In the radiation detector using the flexible base material, the base material is deflected. Therefore, it may be difficult to handle, and improvement in handleability is desired. In particular, in a case where the cable is connected to the terminal and in a case where the base material is deflected, it may be difficult to connect the cable to the terminal in an appropriate state.

Thus, a technique of suppressing the deflection of the base material in the radiation detector is known. For example, in a technique described in JP2004-296656A, a photoelectric conversion substrate and a support member are fixed by a bonding member in a region other than a connecting part between an electric component and the photoelectric conversion substrate on an outer peripheral part of the photoelectric conversion substrate. In the technique described in JP2004-296656A, the deflection of the photoelectric conversion substrate is suppressed by the support member.

### SUMMARY

Meanwhile, in a case where the cable is connected to the terminal, the heat applied to the base material is propagated to the reinforcing member by performing a heat treatment for connection. A reinforcing member may be deformed by the heat propagated from the base material. For example, in the technique described in JP2004-296656A, there is a concern that the support member may be deformed by the heat treatment in a case where a connection electrode on the photoelectric conversion substrate is thermocompression-bonded.

The present disclosure provides a radiation detector, a radiographic imaging apparatus, and a method of manufacturing a radiation detector, which are excellent in handleability and in which deformation of a reinforcing member caused by heat applied to a terminal is suppressed.

A radiation detector of a first aspect of the present disclosure comprises a substrate in which a plurality of pixels that accumulate electric charges generated in response to light converted from radiation are formed in a pixel region of a first surface of a flexible base material and the first surface is provided with a terminal for electrically connecting a cable; a conversion layer that is provided on a first surface side of the base material and converts the radiation into the light; and a reinforcing member that is provided in a region including at least a facing region, facing the terminal, on a second surface of the base material opposite to the first surface and has super engineering plastic as a material.

Additionally, a radiation detector of a second aspect of the present disclosure comprises a substrate in which a plurality of pixels that accumulate electric charges generated in response to light converted from radiation are formed in a pixel region of a first surface of a flexible base material and the first surface is provided with a terminal for electrically connecting a cable; a conversion layer that is provided on the first surface side of the base material and converts the radiation into the light; and a reinforcing member that is provided in a region including at least a facing region, facing the terminal, on a second surface of the base material opposite to the first surface and has a resin with a continuous operating temperature of 150° C. or higher as a main material.

Additionally, a radiation detector of a third aspect of the present disclosure is the radiation detector of the first or second aspect in which the reinforcing member has at least one of a resin having a sulfonyl group, a resin having a phenylene sulfide structure, a resin having an imide group, a resin having an arylene ether structure and an arylene ketone structure, or a resin having a benzimidazole structure as a main material.

Additionally, a radiation detector of a fourth aspect of the present disclosure is the radiation detector of the first or second aspect in which the reinforcing member includes at least one of polysulfone, polyethersulfone, polyphenylene sulfide, polyetheretherketone, or tetrafluoroethylene-ethylene copolymer as a material.

Additionally, a radiation detector of a fifth aspect of the present disclosure is the radiation detector of the first or second aspect in which the reinforcing member includes at least one of polysulfone, polyethersulfone, polyphenylene sulfide, polyamidoimide, polyetheretherketone, polyimide, polybenzoimidazole, thermoplastic polyimide, or tetrafluoroethylene-ethylene copolymer as a material.

Additionally, a radiation detector of a sixth aspect of the present disclosure is the radiation detector of the first or second aspect in which the reinforcing member includes at least one of polysulfone, polyethersulfone, polyphenylene sulfide, polyamidoimide, polyetheretherketone, polyimide, polybenzoimidazole, thermoplastic polyimide, tetrafluoroethylene-ethylene copolymer, polyphenylsulfone, polyarylate, polyetherimide, liquid crystal polymer, polytetrafluoroethylene, tetrafluoroethylene-hexafluoropropylene copolymer, tetrafluoroethylene-perfluoroalkylvinylether copolymer, polychlorotrifluoroethylene, or polyvinylidene fluoride as a material.

Additionally, a radiation detector of a seventh aspect of the present disclosure is the radiation detector of any one of the first to sixth aspects in which a bending stiffness of the reinforcing member is higher than that of the base material.

Additionally, a radiation detector of an eighth aspect of the present disclosure is the radiation detector of any one of the first to seventh aspects in which the reinforcing member is provided in a region of the second surface including the facing region and a part of a region facing a region where the conversion layer is provided.

Additionally, a radiation detector of a ninth aspect of the present disclosure is the radiation detector of any one of the first to eighth aspects further comprising a reinforcing member that is provided in a region where the reinforcing member is not provided, on the second surface of the base material, and has a higher bending stiffness than that of the base material.

Additionally, a radiographic imaging apparatus according to a tenth aspect of the present disclosure comprises a radiation detector of the present disclosure; and a circuit unit for reading out electric charges accumulated in the plurality of pixels.

Additionally, a method of manufacturing a radiation detector according to an eleventh aspect of the present disclosure comprises a step of forming a substrate in which a flexible base material is provided on a support body, a plurality of pixels that accumulate electric charges generated in response to light converted from radiation are formed in a pixel region of a first surface of the base material, and the first surface is provided with a terminal for electrically connecting a cable; a step of forming a conversion layer that converts the radiation into light on the first surface of the base material; a step of peeling the substrate provided with the conversion layer from the support body; and a step of providing a reinforcing member having super engineering plastic as a material in a region including at least a facing region, facing the terminal, on a second surface of the base material opposite to the first surface.

Additionally, a method of manufacturing a radiation detector according to a twelfth aspect of the present disclosure comprises a step of forming a substrate in which a flexible base material is provided on a support body, a plurality of pixels that accumulate electric charges generated in response to light converted from radiation are formed in a pixel region of a first surface of the base material, and the first surface is provided with a terminal for electrically connecting a cable; a step of providing a conversion layer that converts the radiation into light on the first surface of the base material; a step of peeling the substrate provided with the conversion layer from the support body; and a step of providing a reinforcing member having a resin with a continuous operating temperature of 150° C. or higher as a main material in a region including at least a facing region, facing the terminal, on a second surface of the base material opposite to the first surface.

Additionally, a method of manufacturing a radiation detector according to a thirteenth aspect of the present disclosure is the method of manufacturing a radiation detector according to the eleventh or twelfth aspect further comprising a step of electrically connecting the cable to the terminal after the reinforcing member is provided.

According to the present disclosure, handleability is excellent, and the deformation of the reinforcing member caused by the heat applied to a terminal can be suppressed.

### BRIEF DESCRIPTION OF THE DRAWINGS

Exemplary embodiments according to the technique of the present disclosure will be described in detail based on the following figures, wherein:

FIG. 1 is a block diagram showing an example of the configuration of major parts of an electrical system in a radiographic imaging apparatus of an embodiment,

FIG. 2A is a plan view of an example of a radiation detector according to the embodiment as seen from a first surface side of a base material,

FIG. 2B is a plan view of an example of a radiation detector according to the embodiment as seen from a second surface side of a base material,

FIG. 3A is a cross-sectional view taken along line A-A of the radiation detector shown in FIGS. 2A and 2B,

FIG. 3B is a cross-sectional view taken along line B-B of the radiation detector shown in FIGS. 2A and 2B,

FIG. 4A is a cross-sectional view of an example of the radiographic imaging apparatus according to the embodiment,

FIG. 4B is a cross-sectional view of an example of the radiographic imaging apparatus according to the embodiment,

FIG. 5A is a view for explaining an example of a method of manufacturing the radiographic imaging apparatus of the embodiment,

FIG. 5B is a view for explaining an example of the method of manufacturing the radiographic imaging apparatus of the embodiment,

FIG. 5C is a view for explaining an example of the method of manufacturing the radiographic imaging apparatus of the embodiment,

FIG. 5D is a view for explaining an example of the method of manufacturing the radiographic imaging apparatus of the embodiment,

FIG. 5E is a view for explaining an example of the method of manufacturing the radiographic imaging apparatus of the embodiment,

FIG. 6 is a plan view of an example of a radiation detector of Modification Example 1 as seen from a second surface side of a base material,

FIG. 7 is a cross-sectional view taken along line A-A of the radiation detector shown in FIG. 6,

FIG. 8 is a plan view of another example of a radiation detector of Modification Example 1 as seen from a second surface side of a base material,

FIG. 9 is a cross-sectional view taken along line A-A of the radiation detector shown in FIG. 8,

FIG. 10 is a plan view of another example of a radiation detector of Modification Example 1 as seen from a second surface side of a base material,

FIG. 11 is a cross-sectional view taken along line A-A of the radiation detector shown in FIG. 10,

FIG. 12A is a cross-sectional view taken along line A-A of an example of a radiation detector of Modification Example 2,

FIG. 12B is a cross-sectional view taken along line A-A of another example of a radiation detector of Modification Example 2,

FIG. 12C is a cross-sectional view taken along line A-A of an example of a radiation detector of Modification Example 2,

FIG. 12D is a cross-sectional view taken along line A-A of another example of a radiation detector of Modification Example 2,

FIG. 12E is a cross-sectional view taken along line A-A of another example of a radiation detector of Modification Example 2,

FIG. 13 is a cross-sectional view taken along line A-A of an example of a radiation detector of Modification Example 3,

FIG. 14A is a cross-sectional view taken along line A-A of an example of a radiation detector of Modification Example 4,

FIG. 14B is a cross-sectional view taken along line A-A of another example of a radiation detector of Modification Example 4,

FIG. 15 is a cross-sectional view taken along line A-A of an example of a radiation detector of Modification Example 5,

FIG. 16A is a view illustrating an example of a method of manufacturing a radiographic imaging apparatus of Modification Example 6,

FIG. 16B is a view illustrating an example of the method of manufacturing a radiographic imaging apparatus according to Modification Example 6,

FIG. 16C is a view illustrating an example of the method of manufacturing a radiographic imaging apparatus according to Modification Example 6,

FIG. 16D is a view illustrating an example of a method of manufacturing a radiographic imaging apparatus according to Modification Example 6,

FIG. 16E is a view illustrating an example of a method of manufacturing a radiographic imaging apparatus according to Modification Example 6,

FIG. 16F is a view illustrating an example of a method of manufacturing a radiographic imaging apparatus according to Modification Example 6,

FIG. 16G is a view illustrating an example of a method of manufacturing a radiographic imaging apparatus according to Modification Example 6,

FIG. 16H is a view illustrating an example of a method of manufacturing a radiographic imaging apparatus according to Modification Example 6,

FIG. 17A is a cross-sectional view of an example of a radiographic imaging apparatus of Modification Example 7,

FIG. 17B is a cross-sectional view of another example of the radiographic imaging apparatus of Modification Example 7, and

FIG. 17C is a cross-sectional view of still another example of the radiographic imaging apparatus of Modification Example 7.

### DETAILED DESCRIPTION

Hereinafter, embodiments of the present invention will be described in detail with reference to the drawings. In addition, the present embodiments do not limit the present invention.

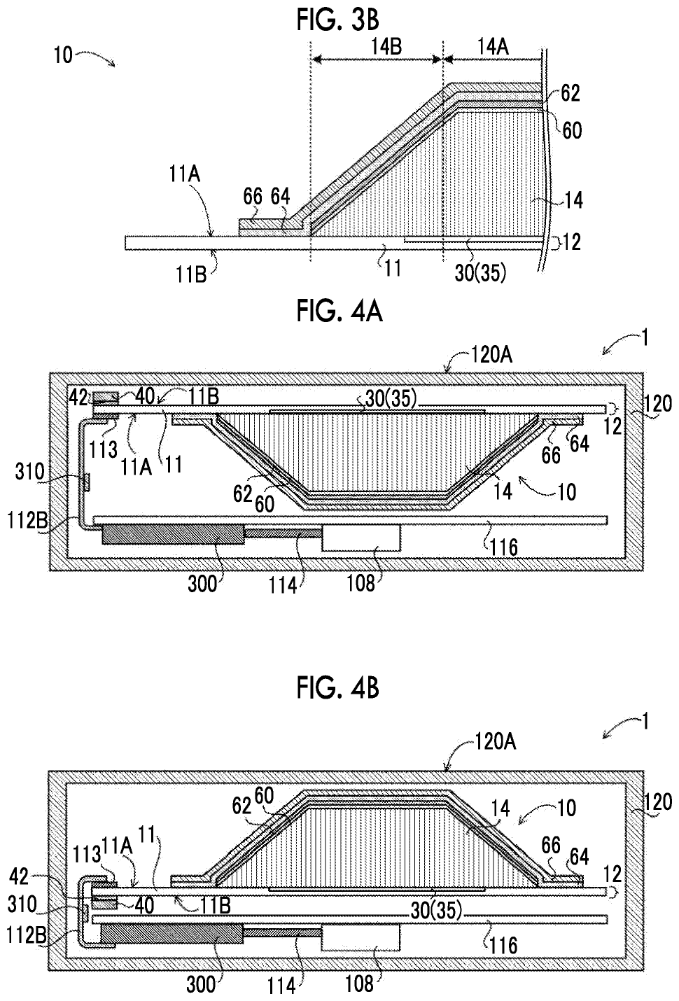

The radiation detector of the present embodiment has a function of detecting radiation transmitted through a subject to output image information representing a radiographic image of the subject. The radiation detector of the present embodiment comprises a sensor substrate and a conversion layer that converts radiation into light (refer to a sensor substrate 12 and a conversion layer 14 of the radiation detector 10 in FIGS. 3A and 3B). The sensor substrate 12 of the present embodiment is an example of a substrate of the present disclosure.

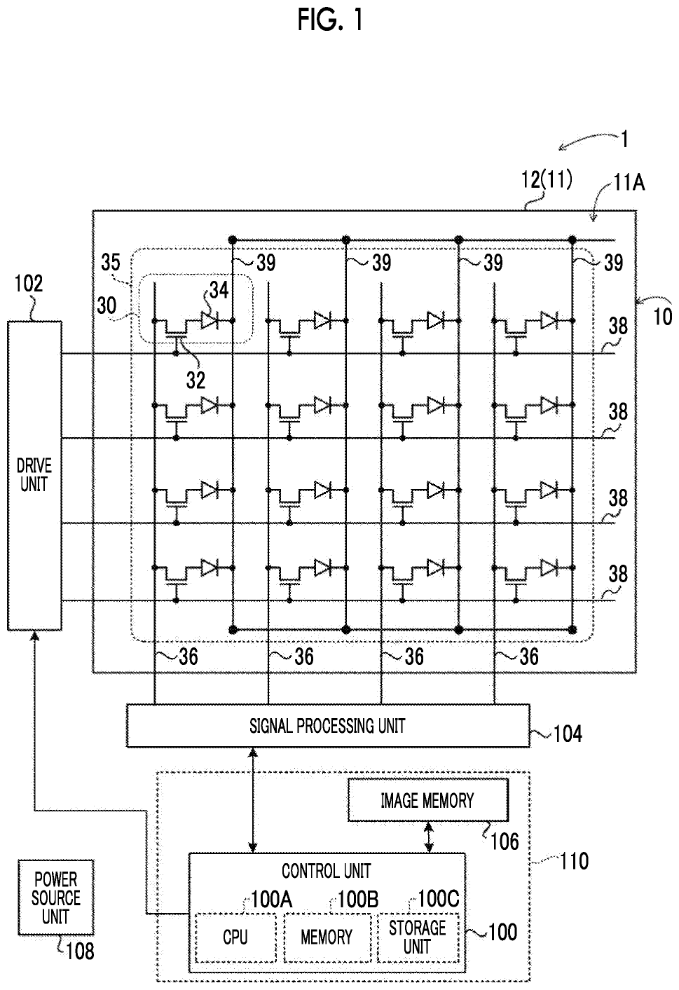

First, the outline of an example of the configuration of an electrical system in a radiographic imaging apparatus of the present embodiment will be described with reference to FIG. 1. FIG. 1 is a block diagram showing an example of the configuration of major parts of the electrical system in the radiographic imaging apparatus of the present embodiment.

As shown in FIG. 1, the radiographic imaging apparatus 1 of the present embodiment comprises the radiation detector 10, a control unit 100, a drive unit 102, a signal processing unit 104, an image memory 106, and a power source unit 108. At least one of the control unit 100, the drive unit 102, or the signal processing unit 104 of the present embodiment is an example of a circuit unit of the present disclosure. Hereinafter, the control unit 100, the drive unit 102, and the signal processing unit 104 are collectively referred to as the “circuit unit”.

The radiation detector 10 comprises a sensor substrate 12 and a conversion layer 14 (refer to FIGS. 3A and 3B) that converts radiation into light. The sensor substrate 12 comprises a flexible base material 11, and a plurality of pixels 30 provided on a first surface 11A of the base material 11. In addition, in the following description, the plurality of pixels 30 may be simply referred to as “pixels 30”.

As shown in FIG. 1, each pixel 30 of the present embodiment comprises a sensor unit 34 that generates and accumulates electric charges in response to the light converted by the conversion layer, and a switching element 32 that reads out the electric charges accumulated in the sensor unit 34. In the present embodiment, as an example, a thin film transistor (TFT) is used as the switching element 32. For that reason, in the following description, the switching element 32 is referred to as a “TFT 32”. In the present embodiment, a layer in which the pixels 30 are formed on the first surface 11A of the base material 11 is provided as a layer that is formed with the sensor unit 34 and the TFT 32 and is planarized.

The pixels 30 are two-dimensionally disposed in one direction (a scanning wiring direction corresponding to a transverse direction of FIG. 1, hereinafter referred to as a “row direction”), and a direction intersecting the row direction (a signal wiring direction corresponding to the longitudinal direction of FIG. 1, hereinafter referred as a “column direction”) in a pixel region 35 of the sensor substrate 12. Although an array of the pixels 30 is shown in a simplified manner in FIG. 1, for example, 1024×1024 pixels 30 are arranged in the row direction and the column direction.

Additionally, a plurality of scanning wiring lines 38, which are provided for respective rows of the pixels 30 to control switching states (ON and OFF) of the TFTs 32, and a plurality of signal wiring lines 36, which are provided for respective columns of the pixels 30 and from which electric charges accumulated in the sensor units 34 are read, are provided in a mutually intersecting manner in the radiation detector 10. Each of the plurality of scanning wiring lines 38 is connected to the drive unit 102 via a flexible cable 112A, and thereby, a drive signal for driving the TFT 32 output from the drive unit 102 to control the switching state thereof flows through each of the plurality of scanning wiring lines 38. Additionally, the plurality of signal wiring lines 36 are electrically connected to the signal processing unit 104 via the flexible cable 112B, respectively, and thereby, electric charges read from the respective pixels 30 are output to the signal processing unit 104 as electrical signals. The signal processing unit 104 generates and outputs image data according to the input electrical signals. In addition, the flexible cable 112 of the present embodiment is an example of a cable of the present disclosure. Additionally, in the present embodiment, the term “connection” with respect to the flexible cable 112 means an electrical connection.

The control unit 100 to be described below is connected to the signal processing unit 104, and the image data output from the signal processing unit 104 is sequentially output to the control unit 100. The image memory 106 is connected to the control unit 100, and the image data sequentially output from the signal processing unit 104 is sequentially stored in the image memory 106 under the control of the control unit 100. The image memory 106 has a storage capacity capable of storing image data equivalent to a predetermined number of sheets, and whenever radiographic images are captured, image data obtained by the capturing is sequentially stored in the image memory 106.

The control unit 100 comprises a central processing unit (CPU) 100A, a memory 100B including a read only memory (ROM), a random access memory (RAM), and the like, and a nonvolatile storage unit 100C, such as a flash memory. An example of the control unit 100 is a microcomputer or the like. The control unit 100 controls the overall operation of the radiographic imaging apparatus 1.

In addition, in the radiographic imaging apparatus 1 of the present embodiment, the image memory 106, the control unit 100, and the like are formed in a control substrate 110.

Additionally, common wiring lines 39 are provided in a wiring direction of the signal wiring lines 36 at the sensor units 34 of the respective pixels 30 in order to apply bias voltages to the respective pixels 30. Bias voltages are applied to the respective pixels 30 from a bias power source by electrically connecting the common wiring lines 39 to the bias power source (not shown) outside the sensor substrate 12.

The power source unit 108 supplies electrical power to various elements and various circuits, such as the control unit 100, the drive unit 102, the signal processing unit 104, the image memory 106, and the power source unit 108. In addition, in FIG. 1, an illustration of wiring lines, which connect the power source unit 108 and various elements or various circuits together, is omitted in order to avoid complications.

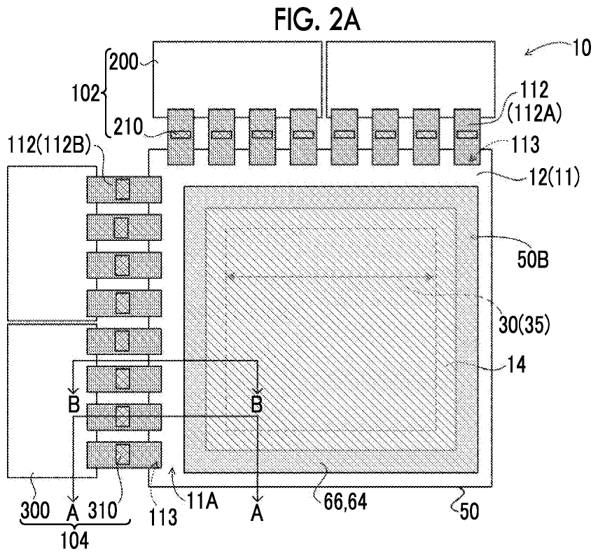

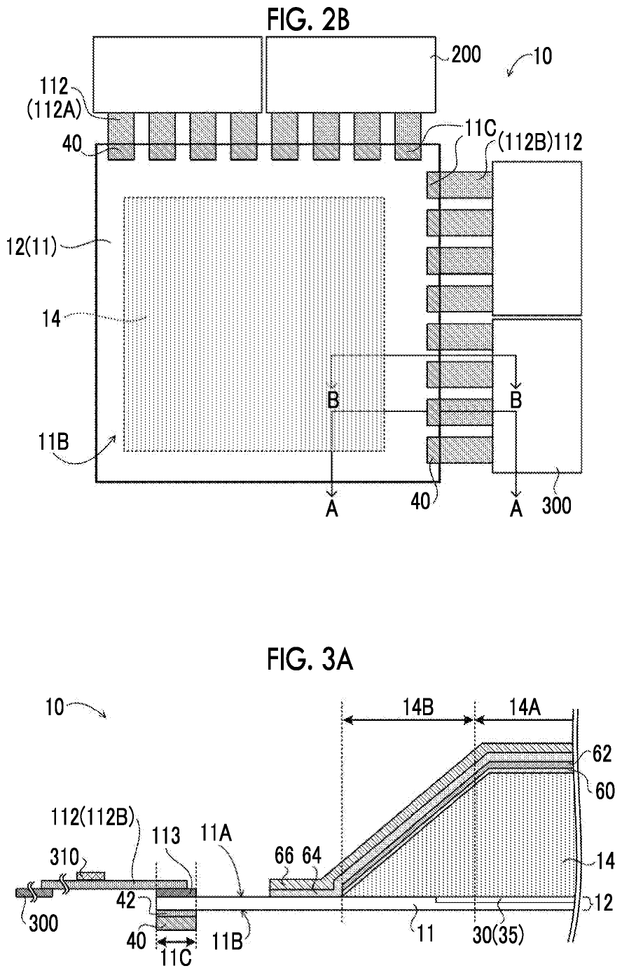

Moreover, the radiation detector 10 will be described in detail. FIG. 2A is an example of a plan view of the radiation detector 10 according to the present embodiment as seen from the first surface 11A side of the base material 11. FIG. 2B is an example of a plan view of the radiation detector 10 according to the present embodiment as seen from the second surface 11B side of the base material 11. Additionally, FIG. 3A is an example of a cross-sectional view taken along line A-A of the radiation detector 10 in FIGS. 2A and 2B. FIG. 3B is an example of a cross-sectional view taken along line B-B of the radiation detector 10 in FIGS. 2A and 2B.

The base material 11 is a resin sheet that has flexibility and includes, for example, a plastic such as a polyimide (PI). The thickness of the base material 11 may be a thickness such that desired flexibility is obtained in response to the hardness of a material, the size of the sensor substrate 12, that is, the area of the first surface 11A or a second surface 11B, and the like. In the case of a rectangular base material 11 alone, an example having flexibility indicates one in which the base material 11 hangs down (becomes lower than the height of the fixed side) 2 mm or more due to the gravity of the base material 11 resulting from its own weight at a position 10 cm away from the fixed side with one side of the base material 11 fixed. As a specific example in a case where the base material 11 is the resin sheet, the thickness thereof may be 5 μm to 125 μm, and the thickness thereof may be more preferably 20 μm to 50 μm.

In addition, the base material 11 has characteristics capable of withstanding the manufacture of the pixels 30 and has characteristics capable of withstanding the manufacture of amorphous silicon TFT (a-Si TFT) in the present embodiment. As such a characteristic of the base material 11, it is preferable that the coefficient of thermal expansion (CTE) at 300° C. to 400° C. is about the same as that of amorphous silicon (Si) wafer (for example, ±5 ppm/K), specifically, the coefficient of thermal expansion is preferably 20 ppm/K or less. Additionally, as the thermal shrinkage rate of the base material 11, it is preferable that the thermal shrinkage rate at 400° C. is 0.5% or less with the thickness being 25 μm. Additionally, it is preferable that the elastic modulus of the base material 11 does not have a transition point that general PI has, in a temperature region of 300° C. to 400° C., and the elastic modulus at 500° C. is 1 GPa or more.

Additionally, it is preferable that the base material 11 of the present embodiment has a fine particle layer containing inorganic fine particles having an average particle diameter of 0.05 μm or more and 2.5 μm or less, which absorbs backscattered rays by itself in order to suppress backscattered rays. In addition, as the inorganic fine particles, in the case of the resinous base material 11, it is preferable to use an inorganic substance of which the atomic number is larger than the atoms constituting the organic substance that is the base material 11 and is 30 or less. Specific examples of such fine particles include SiO₂ that is an oxide of Si having an atomic number of 14, MgO that is an oxide of Mg having an atomic number of 12, Al₂O₃ that is an oxide of Al having an atomic number of 13, TiO₂ that is an oxide of Ti having an atomic number of 22, and the like. A specific example of the resin sheet having such characteristics is XENOMAX (registered trademark).

In addition, the above thicknesses in the present embodiment were measured using a micrometer. The coefficient of thermal expansion was measured according to JIS K7197:1991. In addition, the measurement was performed by cutting out test pieces from a main surface of the base material 11 while changing the angle by 15 degrees, measuring the coefficient of thermal expansion of each of the cut-out test pieces, and setting the highest value as the coefficient of thermal expansion of the base material 11. The coefficient of thermal expansion is measured at intervals of 10° C. between −50° C. and 450° C. in a machine direction (MD) and a transverse direction (TD), and (ppm/° C.) is converted to (ppm/K). For the measurement of the coefficient of thermal expansion, the TMA4000S apparatus made by MAC Science Co., Ltd. is used, sample length is 10 mm, sample width is 2 mm, initial load is 34.5 g/mm², temperature rising rate is 5° C./min, and the atmosphere is in argon.

The base material 11 having desired flexibility is not limited to a resinous material such as the resin sheet. For example, the base material 11 may be a glass substrate or the like having a relatively small thickness. As a specific example of a case where the base material 11 is the glass substrate, generally, in a size of about 43 cm on a side, the glass substrate has flexibility as long as the thickness is 0.3 mm or less. Therefore, any desired glass substrate may be used as long as the thickness is 0.3 mm or less.

As shown in FIGS. 2A, 3A, and 3B, the plurality of pixels 30 are provided on the first surface 11A of the base material 11. In the present embodiment, a region on the first surface 11A of the base material 11 where the pixels 30 are provided is the pixel region 35.

Additionally, the conversion layer 14 is provided on the first surface 11A of the base material 11. The conversion layer 14 of the present embodiment covers the pixel region 35. In the present embodiment, a scintillator including CsI (cesium iodide) is used as an example of the conversion layer 14. It is preferable that such a scintillator includes, for example, CsI:Tl (cesium iodide to which thallium is added) or CsI:Na (cesium iodide to which sodium is added) having an emission spectrum of 400 nm to 700 nm at the time of X-ray radiation. In addition, the emission peak wavelength in a visible light region of CsI:Tl is 565 nm.

In a case where the conversion layer 14 is formed by the vapor-phase deposition method, as shown in FIGS. 3A and 3B, the conversion layer 14 is formed with an inclination such that the thickness thereof gradually decreases toward an outer edge thereof. In the following, a central region 14A of the conversion layer 14 where the thickness in a case where manufacturing errors and measurement errors are neglected can be considered to be substantially constant is referred to as a central part. Additionally, an outer peripheral region of the conversion layer 14 having a thickness of, for example, 90% or less of the average thickness of the central region 14A of the conversion layer 14 is referred to as a peripheral edge part 14B. That is, the conversion layer 14 has an inclined surface that is inclined with respect to the sensor substrate 12 at the peripheral edge part 14B. In addition, in the following, for convenience of description, in a case where “upper” or “lower” are mentioned on the sensor substrate 12, the conversion layer 14 is used as a reference, the side of the conversion layer 14 facing with the sensor substrate 12 is referred to as “lower”, and the opposite side is referred to as “upper”. For example, the conversion layer 14 is provided on the sensor substrate 12, and the inclined surface of the peripheral edge part 14B of the conversion layer 14 is inclined in a state where the conversion layer 14 gradually expands from the upper side to the lower side.

Additionally, as shown in FIGS. 3A and 3B, a pressure-sensitive adhesive layer 60, a, reflective layer 62, an adhesive layer 64, and a protective layer 66 are provided on the conversion layer 14 of the present embodiment.

The pressure-sensitive adhesive layer 60 covers the entire surface of the conversion layer 14. The pressure-sensitive adhesive layer 60 has a function of fixing the reflective layer 62 to the conversion layer 14. The pressure-sensitive adhesive layer 60 preferably has optical transmittance. As materials of the pressure-sensitive adhesive layer 60, for example, an acrylic pressure sensitive adhesive, a hot-melt pressure sensitive adhesive, and a silicone adhesive can be used. Examples of the acrylic pressure sensitive adhesive include urethane acrylate, acrylic resin acrylate, epoxy acrylate, and the like. Examples of the hot-melt pressure sensitive adhesive include thermoplastics, such as ethylene-vinyl acetate copolymer resin (EVA), ethylene-acrylate copolymer resin (EAA), ethylene-ethyl acrylate copolymer resin (EEA), and ethylene-methyl methacrylate copolymer (EMMA). The thickness of the pressure-sensitive adhesive layer 60 is preferably 2 μm or more and 7 μm or less. By setting the thickness of the pressure-sensitive adhesive layer 60 to 2 μm or more, the effect of fixing the reflective layer 62 on the conversion layer 14 can be sufficiently exhibited. Moreover, the risk of forming an air layer between the conversion layer 14 and the reflective layer 62 can be suppressed. When an air layer is formed between the conversion layer 14 and the reflective layer 62, there is a concern that multiple reflections may be caused in which the light emitted from the conversion layer 14 repeats reflections between the air layer and the conversion layer 14 and between the air layer and the reflective layer 62. Additionally, by setting the thickness of the pressure-sensitive adhesive layer 60 to 7 μm or less, it is possible to suppress a decrease in modulation transfer function (MTF) and detective quantum efficiency (DQE).

The reflective layer 62 covers the entire surface of the pressure-sensitive adhesive layer 60. The reflective layer 62 has a function of reflecting the light converted by the conversion layer 14. The material of the reflective layer 62 is preferably made of a resin material containing a metal or a metal oxide. As the material of the reflective layer 62, for example, white PET (Polyethylene terephthalate), TiO₂, Al₂O₃, foamed white PET, specular reflective aluminum, and the like can be used. White PET is obtained by adding a white pigment such as TiO₂ or barium sulfate to PET, and foamed white PET is white PET having a porous surface. Additionally, as the material of the reflective layer 62, a laminated film of a resin film and a metal film may be used. Examples of the laminated film of the resin film and the metal film include an Alpet (registered trademark) sheet in which aluminum is laminated by causing an aluminum foil to adhere to an insulating sheet (film) such as polyethylene terephthalate. The thickness of the reflective layer 62 is preferably 10 μm or more and 40 μm or less. In this way, by comprising the reflective layer 62 on the conversion layer 14, the light converted by the conversion layer 14 can be efficiently guided to the pixels 30 of the sensor substrate 12.

The adhesive layer 64 covers the entire surface of the reflective layer 62. An end part of the adhesive layer 64 extends to the first surface 11A of the base material 11. That is, the adhesive layer 64 adheres to the base material 11 of the sensor substrate 12 at the end part thereof. The adhesive layer 64 has a function of fixing the reflective layer 62 and the protective layer 66 to the conversion layer 14. As the material of the adhesive layer 64, the same material as the material of the pressure-sensitive adhesive layer 60 can be used, but the adhesive force of the adhesive layer 64 is preferably larger than the adhesive force of the pressure-sensitive adhesive layer 60.

The protective layer 66 is provided in a state where the protective layer covers the entire conversion layer 14 and the end part thereof covers a part of the sensor substrate 12. The protective layer 66 functions as a moisture proof film that prevents moisture from entering the conversion layer 14. As the material of the protective layer 66, for example, organic films containing organic materials such as PET, polyphenylene sulfide (PPS), oriented polypropylene (OPP: biaxially oriented polypropylene film), polyethylene naphthalate (PEN), and PI, and Parylene (registered trademark) can be used. Additionally, as the protective layer 66, a laminated film of a resin film and a metal film may be used. Examples of the laminated film of the resin film and the metal film include ALPET (registered trademark) sheets.

Meanwhile, as shown in FIGS. 2A, 3A, and 3B, a plurality (16 in FIG. 2A) of the terminals 113 are provided on an outer edge part of the first surface 11A of the base material 11. An anisotropic conductive film or the like is used as the terminals 113. As shown in FIGS. 2A, 3A, and 3B, the flexible cable 112 is electrically connected to each of the plurality of terminals 113. Specifically, as shown in FIG. 2A, the flexible cable 112A is thermocompression-bonded to each of the plurality of (eight in FIG. 2A) terminals 113 provided on one side of the base material 11. The flexible cable 112A is a so-called chip on film (COF), and a driving integrated circuit (IC) 210 is mounted on the flexible cable 112A. The driving IC 210 is connected to each of a plurality of signal lines included in the flexible cable 112A. In addition, in the present embodiment, the flexible cable 112A and the flexible cable 112B to be described below are simply referred to as “flexible cable 112” in a case where the cables are collectively referred to without distinction.

The other end of the flexible cable 112A opposite to the one end electrically connected to the terminal 113 of the sensor substrate 12 is electrically connected to the driving substrate 200. As an example, in the present embodiment, the plurality of signal lines included in the flexible cable 112A are thermocompression-bonded to the driving substrate 200 and thereby electrically connect to circuits and elements (not shown) mounted on the driving substrate 200. In addition, the method of electrically connecting the driving substrate 200 and the flexible cable 112A is not limited to the present embodiment. For example, a configuration may be adopted in which the driving substrate 200 and the flexible cable 112A are electrically connected by a connector. Examples of such a connector include a zero insertion force (ZIF) structure connector and a Non-ZIF structure connector.

The driving substrate 200 of the present embodiment is a flexible printed circuit board (PCB), which is a so-called flexible substrate. Additionally, circuit components (not shown) mounted on the driving substrate 200 are components mainly used for processing digital signals (hereinafter, referred to as “digital components”). Digital components tend to have a relatively smaller area (size) than analog components to be described below. Specific examples of the digital components include digital buffers, bypass capacitors, pull-up/pull-down resistors, damping resistors, electromagnetic compatibility (EMC) countermeasure chip components, power source ICs, and the like. In addition, the driving substrate 200 may not be necessarily a flexible substrate and may be a non-flexible rigid substrate or a rigid flexible substrate.

In the present embodiment, the drive unit 102 is realized by the driving substrate 200 and the driving IC 210 mounted on the flexible cable 112A. In addition, the driving IC 210 includes, among various circuits and elements that realize the drive unit 102, circuits different from the digital components mounted on the driving substrate 200.

Meanwhile, the flexible cable 112B is electrically connected to each of the plurality (eight in FIG. 2A) of terminals 113 provided on a side intersecting with one side of the base material 11 to which the flexible cable 112A is electrically connected. Similar to the flexible cable 112A, the flexible cable 112B is a so-called chip on film (COF), and a signal processing IC 310 is mounted on the flexible cable 112B. The signal processing IC 310 is connected to a plurality of signal lines (not shown) included in the flexible cable 112B.

The other end of the flexible cable 112B opposite to one end electrically connected to the terminal 113 of the sensor substrate 12 is electrically connected to the signal processing substrate 300. As an example, in the present embodiment, the plurality of signal lines included in the flexible cable 112B are thermocompression-bonded to the signal processing substrate 300 and thereby connected to the circuits and elements (not shown) mounted on the signal processing substrate 300. In addition, the method of electrically connecting the signal processing substrate 300 and the flexible cable 112B is not limited to the present embodiment. For example, a configuration may be adopted in which the signal processing substrate 300 and the cable 112B are electrically connected by a connector. Examples of such a connector include a connector having a ZIF structure, a connector having a Non-ZIF structure, and the like. Additionally, the method of electrically connecting the flexible cable 112A and the driving substrate 200 and the method of electrically connecting the flexible cable 112B and the signal processing substrate 300 may be the same or different. For example, a configuration may be adopted in which the flexible cable 112A and the driving substrate 200 are electrically connected by thermocompression bonding, and the flexible cable 112B and the signal processing substrate 300 are electrically connected by a connector.

The signal processing substrate 300 of the present embodiment is a flexible PCB, which is a so-called flexible substrate, similarly to the above-described driving substrate 200. Circuit components (not shown) mounted on the signal processing substrate 300 are components mainly used for processing analog signals (hereinafter referred to as “analog components”). Specific examples of the analog components include charge amplifiers, analog-to-digital converters (ADCs), digital-to-analog converters (DAC), and power source ICs. Additionally, the circuit components of the present embodiment also include coils around a power source, which has a relatively large component size, and large-capacity smoothing capacitors. In addition, the signal processing substrate 300 may not be necessarily a flexible substrate and may be a non-flexible rigid substrate or a rigid flexible substrate.

In the present embodiment, the signal processing unit 104 is realized by the signal processing substrate 300 and the signal processing IC 310 mounted on the flexible cable 112B. In addition, the signal processing IC 310 includes, among various circuits and elements that realize the signal processing unit 104, circuits different from the analog components mounted on the signal processing substrate 300.

In addition, in FIGS. 2A and 2B, a configuration in which a plurality of (two) the driving substrates 200 and a plurality of (two) the signal processing substrates 300 are provided has been described. However, the number of driving substrates 200 and the number of signal processing substrates 300 are not limited to those shown in FIGS. 2A and 2B. For example, a configuration may be adopted in which at least one of the driving substrate 200 or the signal processing substrate 300 may be a single substrate.

Meanwhile, as shown in FIG. 3A, in the radiation detector 10 of the present embodiment, the flexible cable 112 is thermocompression-bonded to the terminal 113, and thereby the flexible cable 112 is electrically connected to the terminal 113. In addition, although FIG. 3A is a view showing an example of a structure relating to the electrical connection between the flexible cable 112B and the radiation detector 10, a structure related to the electrical connection between the flexible cable 112A and the radiation detector 10 of the present embodiment is also the same as the configuration shown in FIG. 3A.

Additionally, as shown in FIGS. 2B, 3A, and 3B, a reinforcing member 40 is provided on the second surface 11B side of the base material 11 in the sensor substrate 12 of the radiation detector 10 of the present embodiment. Specifically, the reinforcing member 40 of the present embodiment is provided in a facing region 11C, facing the terminal 113, of the second surface 11B of the base material 11. In addition, the reinforcing member 40 may be provided in a region including at least the facing region 11C on the second surface 11B of the base material 11.

As described above, in the step of electrically connecting the flexible cable 112 to the terminal 113 of the base material 11, in a case where a region provided with the terminal 113 of the base material 11 is deflected, for example, problems may occur, such that the terminal 113 and the flexible cable 112 are connected to each other in a deviated state. Thus, in the radiation detector 10 of the present embodiment, the stiffness of at least a region where the terminal 113 of the base material 11 is provided is reinforced by the reinforcing member 40. For that reason, the reinforcing member 40 has a function of reinforcing the stiffness of the base material 11. The reinforcing member 40 of the present embodiment is higher in bending stiffness than the base material 11, and the dimensional change (deformation) thereof with respect to a force applied in a direction perpendicular to the surface opposite to the conversion layer 14 is smaller than the dimensional change thereof with respect to a force applied in the direction perpendicular to the second surface 11B of the base material 11.

In addition, the bending stiffness of the reinforcing member 40 is preferably 100 times or more the bending stiffness of the base material 11. Additionally, the thickness of the reinforcing member 40 of the present embodiment is larger than the thickness of the base material 11. For example, in a case where XENOMAX (registered trademark) is used as the base material 11, the thickness of the reinforcing member 40 is preferably about 0.1 mm to 0.25 mm.

From the viewpoint of suppressing the deflection of the base material 11, the reinforcing member 40 preferably has a higher bending stiffness than the base material 11. Specifically, a material having a bending elastic modulus of 150 MPa or more and 5,000 MPa or less is preferably used for the reinforcing member 40 of the present embodiment. In addition, in a case where the bending elastic modulus becomes low, the bending stiffness also becomes low. In order to obtain a desired bending stiffness, the thickness of the reinforcing member 40 should be made large, and the thickness of the entire radiation detector 10 increases. Considering the above-described material of the reinforcing member 40, the thickness of the reinforcing member 40 tends to be relatively large in a case where a bending stiffness exceeding 140,000 Pacm⁴ is to be obtained. For that reason, in view of obtaining appropriate stiffness and considering the thickness of the entire radiation detector 10, the material used for the reinforcing member 40 preferably has a bending elastic modulus of 150 MPa or more and 5,000 MPa or less. Additionally, the bending stiffness of the reinforcing member 40 is preferably 540 Pacm⁴ or more and 280,000 Pacm⁴ or less.

As described above, in a case where the flexible cable 112 is electrically connected to the terminal 113, a heat treatment for thermocompression-bonding the terminal 113 and the flexible cable 112 is performed. By this heat treatment, the heat applied to the base material 11 propagates to the reinforcing member 40. In a case where the reinforcing member 40 is deformed by the propagated heat, for example, the reinforcing member 40 may be peeled from the base material 11. Additionally, for example, the base material 11 may also be deformed to follow the deformation of the reinforcing member 40, and the electrical connection between the flexible cable 112 and the terminal 113 may be cut off or the quality of a radiographic image obtained by the radiation detector 10 may be affected.

The heat applied to the base material 11 due to the heat treatment mainly tends to propagate from the facing region 11C of the second surface 11B to the reinforcing member 40. Thus, in the radiation detector 10 of the present embodiment, the reinforcing member 40 having excellent heat resistance is provided in the facing region 11C of the second surface 11B of the base material 11. In this way, in the radiation detector 10 of the present embodiment, in a case where the flexible cable 112 is pressure-bonded to the terminal 113 of the base material 11, the reinforcing member 40 that is not deformed by the heat applied to the base material 11 or the reinforcing member 40 in which the amount of deformation caused by the heat is within a permissible range is provided in the facing region 11C of the second surface 11B of the base material 11.

It is preferable that the material of the reinforcing member 40 that satisfies the above heat resistance is a material of which a main component is a material in which a continuous operating temperature based on UL 746B regulations of the UL standard by the American Insurer Safety Testing Laboratory is 150° C. or higher. Alternatively, it is preferable that the material of the reinforcing member 40 that satisfies the above heat resistance is a material having super engineering plastic (hereinafter referred to as “super engineering plastic”) as a main component. Alternatively, it is preferable that the above material is a material having a resin having a sulfonyl group, a resin having a phenylene sulfide structure, a resin having an imide group, a resin having an arylene ether structure and an arylene ketone structure, a resin having a benzimidazole structure, and the like as a main component.

Specifically, from the viewpoint of bending stiffness and heat resistance, the materials of the reinforcing member 40 of the present embodiment include at least one of polysulfone (PSU, PSF), polyethersulfone (PES), polyphenylene sulfide (PPS), polyamidoimide (PAI), polyetheretherketone (PEEK), polyimide (PI), polybenzoimidazole (PBI), thermoplastic polyimide (TPI), tetrafluoroethylene-ethylene copolymer (ETFE), polyphenylsulfone (PPSU, PPSF), polyarylate (PAR), polyetherimide (PEI), liquid crystal polymer (LCP), polytetrafluoroethylene (PTFE), tetrafluoroethylene-hexafluoropropylene copolymer (FEP), tetrafluoroethylene-perfluoroalkylvinylether polymer (PFA), polychlorotrifluoroethylene (PCTFE), or polyvinylidene fluoride (PVDF).

Moreover, among these, it is preferable that the main materials of the reinforcing member 40 includes at least one of polysulfone (PSU), polyethersulfone (PES), polyphenylene sulfide (PPS), polyamidoimide (PAI), polyetheretherketone (PEEK), polyimide (PI), polybenzoimidazole (PBI), thermoplastic polyimide (TPI), and tetrafluoroethylene-ethylene copolymer (ETFE). Moreover, considering impact resistance and the like, it is more preferable that the main materials of the reinforcing member 40 include at least one of polysulfone (PSU), polyethersulfone (PES), polyphenylene sulfide (PPS), polyetheretherketone (PEEK), or tetrafluoroethylene-ethylene copolymer (ETFE) as a material.

Moreover, the radiographic imaging apparatus 1 will be described in detail. FIG. 4A is an example of a cross-sectional view of a radiographic imaging apparatus 1 in a case where the radiation detector 10 of the present embodiment is applied to an irradiation side sampling (ISS) type in which radiation is emitted from the second surface 11B side of the base material 11. Additionally, FIG. 4B is an example of a cross-sectional view of the radiographic imaging apparatus 1 in a case where the radiation detector 10 of the present embodiment is applied to the penetration side sampling (PSS) type in which radiation is emitted from the conversion layer 14 side.

The radiographic imaging apparatus 1 formed of the above radiation detector 10 is used while being housed in a housing 120, as shown in FIGS. 4A and 4B. As shown in FIGS. 4A and 4B, the radiation detector 10, the power source unit 108, and the circuit unit such as the signal processing substrate 300 are provided side by side in an incidence direction of radiation within the housing 120. The radiation detector 10 of FIG. 4A is disposed in a state where the second surface 11B of the base material 11 faces a top plate on an irradiation surface 120A side of the housing 120 that is irradiated with the radiation transmitted through a subject. More specifically, the reinforcing member 40 is disposed so as to face the top plate on the irradiation surface 120A side of the housing 120. Additionally, the radiation detector 10 of FIG. 4B is disposed in a state where the first surface 11A side of the base material 11 faces the top plate on the irradiation surface 120A side of the housing 120. More specifically, an upper surface of the conversion layer 14 is disposed so as to face the top plate on the irradiation surface 120A side of the housing 120.

Additionally, a middle plate 116 is further provided on a side from which the radiation transmitted through the radiation detector 10 is emitted, within the housing 120 as shown in FIGS. 4A and 4B. The middle plate 116 is, for example, an aluminum or copper sheet. The copper sheet does not easily generate secondary radiation due to incident radiation, and therefore, has a function of preventing scattering to the rear side, that is, the conversion layer 14 side. In addition, it is preferable that the middle plate 116 covers at least an entire surface of the conversion layer 14 from which radiation is emitted, and covers the entire conversion layer 14. Additionally, a circuit unit such as a signal processing substrate 300 is fixed to the middle plate 116.

The housing 120 is preferably lightweight, has a low absorbance of radiation, particularly X-rays, and has a high stiffness, and is more preferably made of a material having a sufficiently high elastic modulus. As the material of the housing 120, it is preferable to use a material having a bending modulus of elasticity of 10,000 MPa or more. As the material of the housing 120, carbon or carbon fiber reinforced plastics (CFRP) having a bending modulus of elasticity of about 20,000 MPa to 60,000 MPa can be suitably used.

In the capturing of a radiographic image by the radiographic imaging apparatus 1, a load from a subject is applied to the irradiation surface 120A of the housing 120. In a case where the stiffness of the housing 120 is insufficient, there are concerns that problems may occur such that the sensor substrate 12 is deflected due to the load from the subject and the pixels 30 are damaged. By housing the radiation detector 10 inside the housing 120 consisting of a material having a bending modulus of elasticity of 10,000 MPa or more, it is possible to suppress the deflection of the sensor substrate 12 due to the load from the subject.

In addition, the housing 120 may be formed of different materials for the irradiation surface 120A of the housing 120 and other portions. For example, a portion corresponding to the irradiation surface 120A may be formed of a material having a low radiation absorbance and high stiffness and having a sufficiently high elastic modulus, and the other portions may be formed of a material different from the portion corresponding to the irradiation surface 120A, for example, a material having a lower elastic modulus than the portion of the irradiation surface 120A.

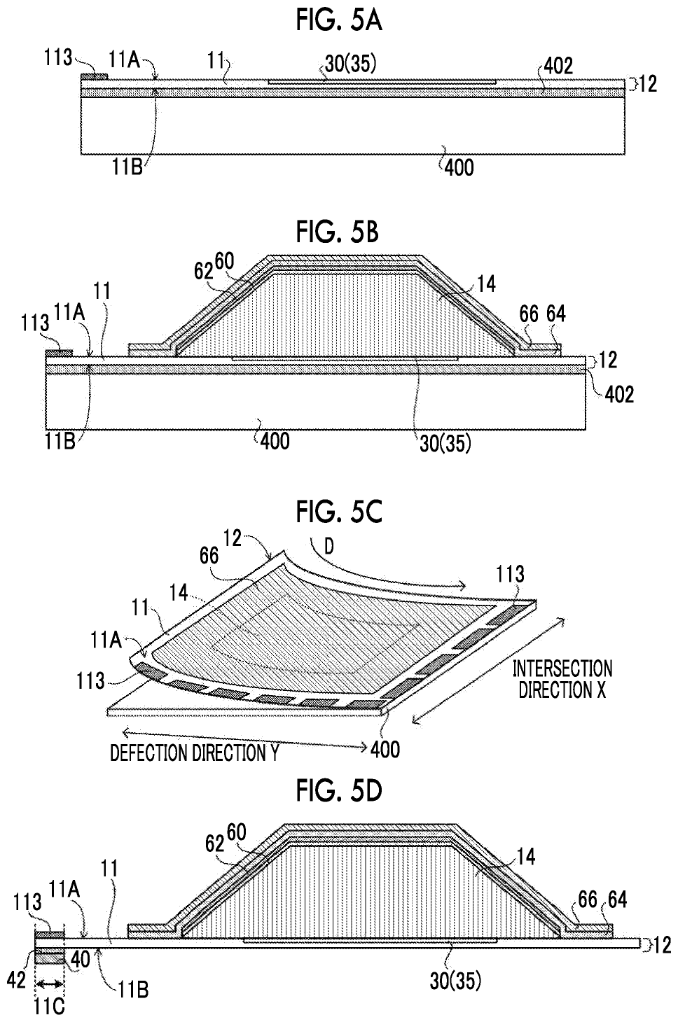

A method of manufacturing the radiographic imaging apparatus 1 of the present embodiment will be described with reference to FIGS. 5A to 5E. In addition, the method of manufacturing the radiographic imaging apparatus 1 of the present embodiment includes a method of manufacturing the radiation detector 10 of the present embodiment.

As shown in FIG. 5A, the base material 11 is provided on a support body 400, such as a glass substrate having a thickness larger than that of the base material 11, via a peeling layer 402, for example in order to form the sensor substrate 12. For example, in a case where the base material 11 is formed by a lamination method, a sheet to be the base material 11 is bonded onto the support body 400. The second surface 11B of the base material 11 is in contact with the peeling layer 402. In addition, the method of forming the base material 11 is not limited to the present embodiment. For example, a configuration may be adopted in which the base material 11 is formed by a coating method.

Moreover, the pixels 30 and terminal 113 are formed on the first surface 11A of the base material 11. The pixel 30 is formed via an undercoat layer (not shown) formed of SiN or the like in the pixel region 35 of the first surface 11A. Additionally, a plurality of the terminals 113 are formed along each of two sides of the base material 11.

Additionally, as shown in FIG. 5B, the conversion layer 14 is formed on a layer on which the pixels 30 are formed (hereinafter, simply referred to as “pixels 30”). In the present embodiment, the conversion layer 14 of CsI is directly formed as a columnar crystal on the sensor substrate 12 by vapor-phase deposition methods, such as a vacuum vapor deposition method, a sputtering method, and a chemical vapor deposition (CVD) method. In this case, the side of the conversion layer 14 in contact with the pixels 30 is a growth-direction base point side of the columnar crystal.

Additionally, unlike the radiation detector 10 of the present embodiment, GOS (Gd₂O₂S:Tb)) or the like may be used as the conversion layer 14 instead of CsI. In this case, for example, the conversion layer 14 can be formed on the sensor substrate 12 by preparing one in which a sheet having GOS dispersed in a binder such as resin is bonded to a support body formed of white PET or the like with a pressure-sensitive adhesive layer or the like, and bonding a side of the GOS on which the support body is not bonded, and the pixel 30 of the sensor substrate 12 to each other with the pressure sensitive adhesive sheet or the like. In addition, the conversion efficiency from radiation to visible light is higher in a case where CsI is used for the conversion layer 14 than in a case where GOS is used.

Moreover, the reflective layer 62 is provided on the conversion layer 14 formed on the sensor substrate 12 via the pressure-sensitive adhesive layer 60. Moreover, the protective layer 66 is provided via the adhesive layer 64.

After that, as shown in FIG. 5C, the sensor substrate 12 provided with the conversion layer 14 is peeled from the support body 400. Hereinafter, this step is referred to as a peeling step. In the case of mechanical peeling, in an example shown in FIG. 5C, a side of the base material 11 of the sensor substrate 12 facing a side on which the terminal 113 is provided is a starting point for the peeling, and the sensor substrate 12 is peeled from the support body 400 by gradually peeling the sensor substrate 12 in the direction of an arrow D shown in FIG. 5C from the support body 400, toward the side where the terminal 113 is provided from the side serving as the starting point.

In addition, it is preferable that the side to be the peeling starting point is a side that intersects the longest side in a case where the sensor substrate 12 is seen in a plan view. In other words, the side in a deflection direction Y in which the deflection is caused by the peeling is preferably the longest side. As an example, in the present embodiment, the peeling starting point is the side opposite to the side to which the flexible cable 112B is electrically connected.

Next, as shown in FIG. 5D, the reinforcing member 40 provided with the pressure sensitive adhesive 42 is bonded to the facing region 11C of the second surface 11B of the base material 11.

Next, as shown in FIG. 5E, the flexible cable 112 is electrically connected to the sensor substrate 12. Specifically, the flexible cable 112 on which the driving IC 210 or the signal processing IC 310 is mounted is thermocompression-bonded to the terminal 113 to electrically connect the terminal 113 and the flexible cable 112. Accordingly, the flexible cable 112 is electrically connected to the sensor substrate 12.

Moreover, by housing the radiation detector 10, the circuit unit, and the like in the housing 120, the radiographic imaging apparatus 1 shown in FIG. 4A or FIG. 4B is manufactured. Specifically, by housing the radiation detector 10 in the housing 120 in a state where the reinforcing member 40 faces the irradiation surface 120A, the radiographic imaging apparatus 1 shown in FIG. 4A is manufactured. Additionally, by housing the radiation detector 10 in the housing 120 in a state where the conversion layer 14 faces the irradiation surface 120A, the radiographic imaging apparatus 1 shown in FIG. 4B is manufactured.

In addition, the configuration and manufacturing method of the radiographic imaging apparatus 1 and the radiation detector 10 are not limited to the above-described form. For example, the configurations shown in the following Modification Examples 1 to 7 may be used. In addition, configurations may be adopted in which the above-described form and respective Modification Examples 1 to 7 are combined appropriately, and the invention is not limited to Modification Examples 1 to 7.

### Modification Example 1

In the present modification example, a modification example of the reinforcing member 40 will be described.

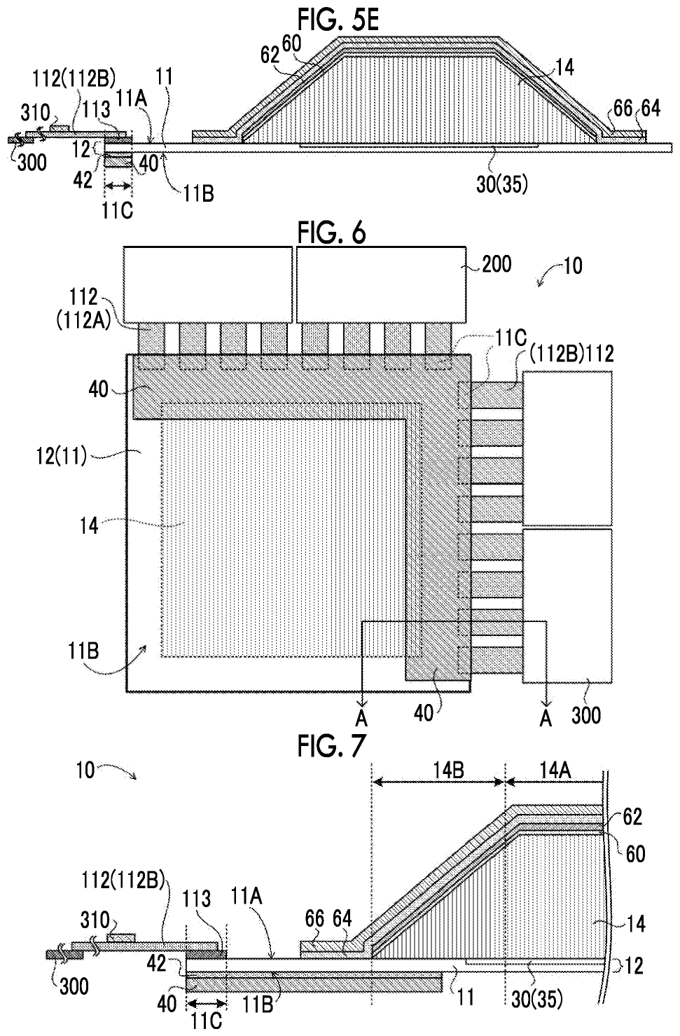

FIG. 6 is an example of a plan view of the radiation detector 10 according to the present modification example as seen from the second surface 11B side of the base material 11. Additionally, FIG. 7 is an example of a cross-sectional view taken along line A-A of the radiation detector 10 in FIG. 6.

As shown in FIG. 6, the reinforcing member 40 of the radiation detector 10 of the present modification example is provided on the entire side of the base material 11 on which the terminal 113 is provided, that is, the entire side having the facing region 11C. Specifically, the reinforcing member 40 is provided on the entire side provided with the facing region 11C of the terminal 113 to which the flexible cable 112A is electrically connected and the entire side provided with the facing region 11C of the terminal 113 to which the flexible cable 112B is electrically connected are provided.

Additionally, as shown in FIGS. 6 and 7, the reinforcing member 40 of the present modification example is provided in a region including the facing region 11C on the second surface 11B and a part of a region facing a region provided with the conversion layer 14. Specifically, the reinforcing member 40 of the present modification example is seamlessly provided on the second surface 11B from the side provided with the facing region 11C to the inside of a region below the peripheral edge part 14B of the conversion layer 14.

By providing the reinforcing member 40 in this way, it is possible to suppress local deflection and non-uniform deflection that occur at a boundary between the region provided with the reinforcing member 40 and a region where the reinforcing member 40 is not provided. In particular, in the vicinity of an outer edge part of the conversion layer 14 in the base material 11, the deflection is likely to occur due to a change in thickness or the like. In contrast, in the radiation detector 10 of the present modification example shown in FIGS. 6 and 7, the reinforcing member 40 is provided on the second surface 11B of the base material 11, which is below the outer edge part of the conversion layer 14. Therefore, it is possible to suppress the deflection of the base material 11 in the vicinity of the outer edge part of the conversion layer 14. In this way, since the deflection of the base material 11 is suppressed, it is possible to suppress the peeling of the conversion layer 14 from the base material 11.

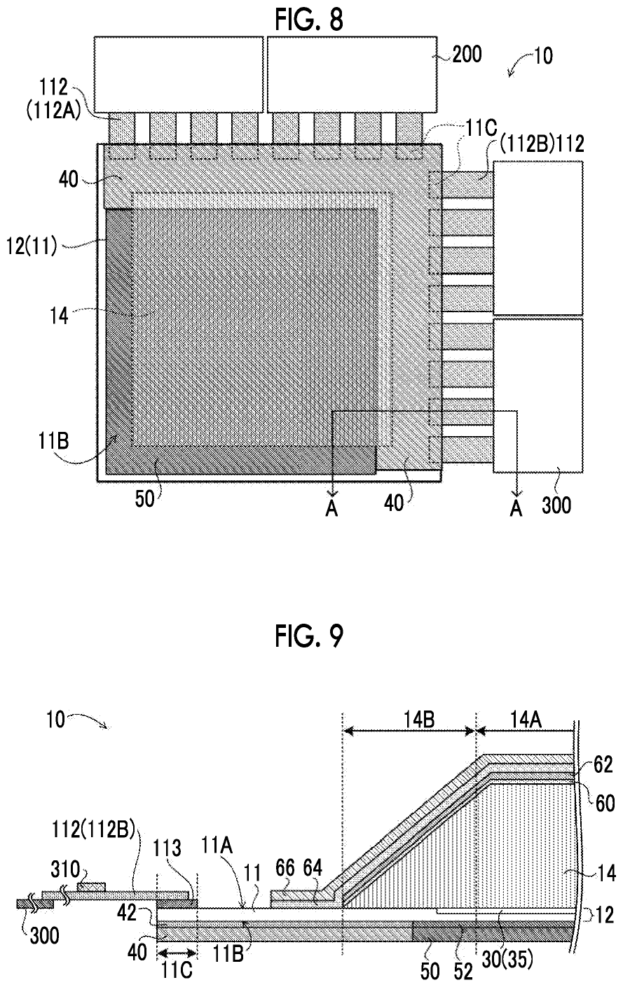

In addition, as shown in FIGS. 8 and 9, a rigid plate 50 may be provided by a pressure sensitive adhesive 52 in the region where the reinforcing member 40 is not provided on the second surface 11B of the base material 11. FIG. 8 is an example of a plan view of the radiation detector 10 according to the present modification example as seen from the second surface 11B side of the base material 11. Additionally, FIG. 9 is an example of a cross-sectional view taken along line A-A of the radiation detector 10 in FIG. 8.

Similar to the reinforcing member 40, the rigid plate 50 has a function of reinforcing the stiffness of the base material 11. The rigid plate 50 of the present embodiment is higher in bending stiffness than the base material 11, and the dimensional change (deformation) thereof with respect to a force applied in a direction perpendicular to the surface opposite to the conversion layer 14 is smaller than the dimensional change thereof with respect to a force applied in the direction perpendicular to the second surface 11B of the base material 11.

In addition, specifically, the bending stiffness of the rigid plate 50 is preferably 100 times or more the bending stiffness of the base material 11. Additionally, the thickness of the rigid plate 50 of the present embodiment is larger than the thickness of the base material 11. For example, in a case where XENOMAX (registered trademark) is used as the base material 11, the thickness of the rigid plate 50 is preferably about 0.1 mm to 0.25 mm. In addition, it is preferable that the thickness of the rigid plate 50 and the thickness of the reinforcing member 40 are the same.

Specifically, a material having a bending elastic modulus of 150 MPa or more and 2,500 MPa or less is preferably used for the rigid plate 50 of the present embodiment. From the viewpoint of suppressing the deflection of the base material 11, the rigid plate 50 preferably has a higher bending stiffness than the base material 11. In addition, in a case where the bending elastic modulus becomes low, the bending stiffness also becomes low. In order to obtain a desired bending stiffness, the thickness of the rigid plate 50 should be made large, and the thickness of the entire radiation detector 10 increases. Considering the above-described material of the rigid plate 50, the thickness of the rigid plate 50 tends to be relatively large in a case where a bending stiffness exceeding 140,000 Pacm⁴ is to be obtained. For that reason, in view of obtaining appropriate stiffness and considering the thickness of the entire radiation detector 10, the material used for the rigid plate 50 preferably has a bending elastic modulus of 150 MPa or more and 2,500 MPa or less. Additionally, the bending stiffness of the rigid plate 50 is preferably 540 Pacm⁴ or more and 140,000 Pacm⁴ or less.

Additionally, the coefficient of thermal expansion of the rigid plate 50 of the present embodiment is preferably closer to the coefficient of thermal expansion of the material of the conversion layer 14, and the ratio of the coefficient of thermal expansion of the rigid plate 50 to the coefficient of thermal expansion of the conversion layer 14 (the coefficient of thermal expansion of the rigid plate 50/the coefficient of thermal expansion of the conversion layer 14) is more preferably 0.5 or more and 2 or less. The coefficient of thermal expansion of such a rigid plate 50 is preferably 30 ppm/K or more and 80 ppm/K or less. For example, in a case where the conversion layer 14 has CsI:Tl as a material, the coefficient of thermal expansion is 50 ppm/K. In this case, examples of materials relatively close to the conversion layer 14 include polyvinyl chloride (PVC) having a coefficient of thermal expansion of 60 ppm/K to 80 ppm/K, acrylic having a coefficient of thermal expansion of 70 ppm/K to 80 ppm/K, PET having a coefficient of thermal expansion of 65 ppm/K to 70 ppm/K, polycarbonate (PC) having a coefficient of thermal expansion of 65 ppm/K, Teflon (registered trademark) having a coefficient of thermal expansion of 45 ppm/K to 70 ppm/K, and the like. Moreover, considering the above-described bending elastic modulus, the material of the rigid plate 50 is more preferably a material containing at least one of PET or PC.

From the viewpoint of elasticity, the rigid plate 50 preferably contains a material having a yield point. In addition, in the present embodiment, the “yield point” means a phenomenon in which the stress rapidly decreases once in a case where the material is pulled, means that the strain is increased without increasing the stress on a curve representing a relationship between the stress and the strain, and indicates the peak of a stress-strain curve in a case where a tensile strength test is performed on the material. Resins having the yield point generally include resins that are hard and strongly sticky, and resins that are soft and strongly sticky and have medium strength. Examples of the hard and strongly sticky resins include PC and the like. Additionally, examples of the resins that are soft and strongly sticky and have medium strength include polypropylene and the like.

In a case where the rigid plate 50 of the present embodiment is a substrate having plastic as a material, the material is preferably a thermoplastic resin for the above-described reasons, and examples thereof include at least one of PC, PET, styrol, acrylic, polyacetase, nylon, polypropylene, acrylonitrile butadiene styrene (ABS), engineering plastics, or polyphenylene ether. In addition, the rigid plate 50 is even more preferably at least one of polypropylene, ABS, engineering plastics, PET, or polyphenylene ether among these, is more preferably at least one of styrol, acrylics, polyacetase, or nylon, and is more preferably at least one of PC or PET.

In this way, in the radiation detector 10 shown in FIGS. 8 and 9, the reinforcing member 40 or the rigid plate 50 is provided on the second surface 11B of the base material 11 to reinforce the entire second surface 11B with the reinforcing member 40 or the rigid plate 50. Accordingly, the bending stiffness of the entire base material 11 can be reinforced.

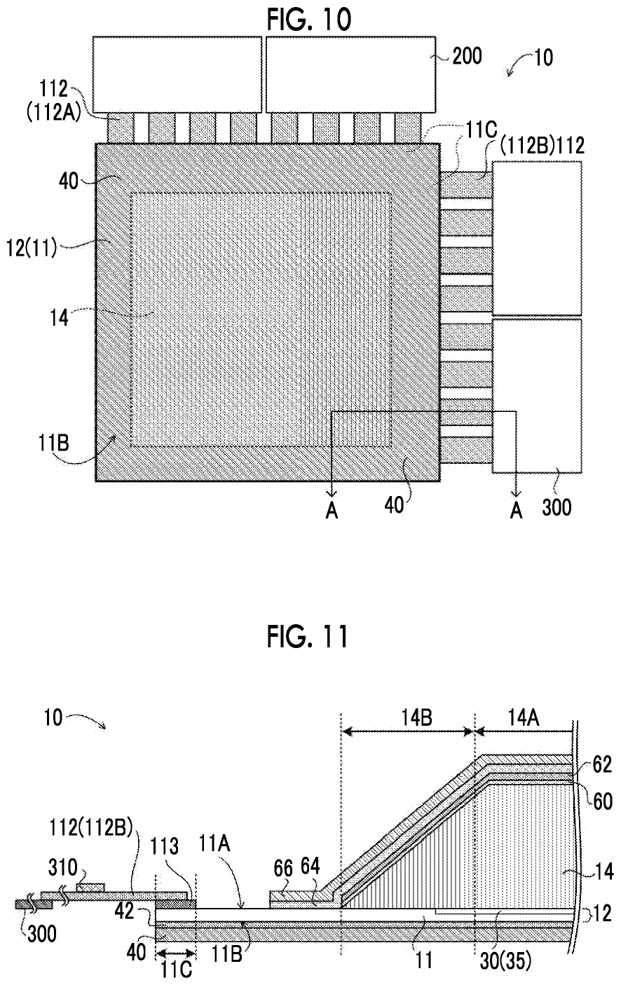

In addition, the reinforcing member 40 may be provided on the second surface 11B of the base material 11, which corresponds to the entire lower side of the conversion layer 14. That is, as shown in FIGS. 10 and 11, the reinforcing member 40 may be provided on the entire second surface 11B of the base material 11 of the radiation detector 10. FIG. 10 is another example of a plan view of the radiation detector 10 according to the present modification example as seen from the second surface 11B side of the base material 11. Additionally, FIG. 11 is an example of a cross-sectional view taken along line A-A of the radiation detector 10 in FIG. 10. As shown in FIG. 10, by providing the reinforcing member 40 on the entire second surface 11B of the base material 11, the bending stiffness of the base material 11 can be further reinforced. Additionally, the local deflection of the base material 11 on the second surface 11B can be further suppressed.

### Modification Example 2

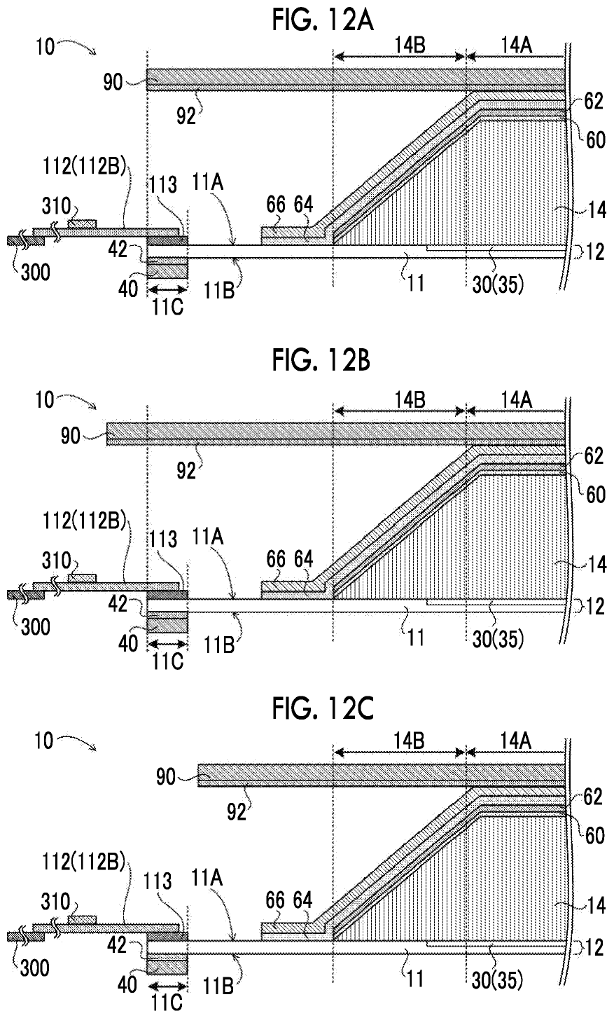

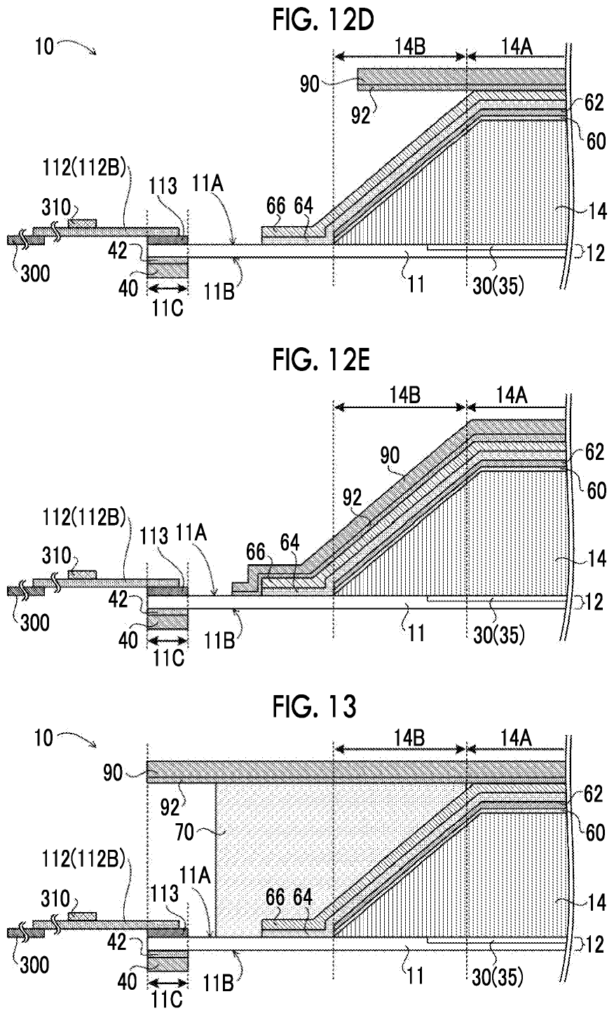

In the present modification example, referring to FIGS. 12A to 12E, the example of a radiation detector in a case where a reinforcing substrate 90 that reinforces the stiffness of the base material 11 is provided on the first surface 11A side of the base material 11 of the sensor substrate 12. 10 will be described. Each of FIGS. 12A to 12E shows an example of a cross-sectional view of a radiation detector 10 of the present modification example, which corresponds to the sectional view taken along the line A-A of the radiation detector 10 shown in FIG. 3A.

As shown in FIG. 12A, a pressure sensitive adhesive 92 and the reinforcing substrate 90 are provided on the conversion layer 14 provided on the first surface 11A of the base material 11.

The reinforcing substrate 90 has a higher bending stiffness than the base material 11, and a dimensional change (deformation) due to a force applied in a direction perpendicular to the surface facing the conversion layer 14 is smaller than a dimensional change due to a force applied in a direction perpendicular to the first surface 11A of the base material 11. Additionally, the thickness of the reinforcing substrate 90 of the present modification example is larger than the thickness of the base material 11.

The preferable characteristics of the reinforcing substrate 90 are the same characteristics as those of the above-described rigid plate 50 in Modification Example 1. The reinforcing substrate 90 of the present modification example preferably uses a material having a bending elastic modulus of 150 MPa or more and 2,500 MPa or less. From the viewpoint of suppressing the deflection of the base material 11, the reinforcing substrate 90 preferably has a higher bending stiffness than the base material 11. In addition, in a case where the bending elastic modulus becomes low, the bending stiffness also becomes low. In order to obtain a desired bending stiffness, the thickness of the reinforcing substrate 90 should be made large, and the thickness of the entire radiation detector 10 increases. Considering the material of the reinforcing substrate 90, the thickness of the reinforcing substrate 90 tends to be relatively large in a case where a bending stiffness exceeding 140,000 Pacm⁴ is to be obtained. For that reason, in view of obtaining appropriate stiffness and considering the thickness of the entire radiation detector 10, the material used for the reinforcing substrate 90 preferably has a bending elastic modulus of 150 MPa or more and 2,500 MPa or less. Additionally, the bending stiffness of the reinforcing substrate 90 is preferably 540 Pacm⁴ or more and 140,000 Pacm⁴ or less.

Additionally, the coefficient of thermal expansion of the reinforcing substrate 90 is preferably closer to the coefficient of thermal expansion of the material of the conversion layer 14, and the ratio of the coefficient of thermal expansion of the reinforcing substrate 90 to the coefficient of thermal expansion of the conversion layer 14 (the coefficient of thermal expansion of the reinforcing substrate 90/the coefficient of thermal expansion of the conversion layer 14) is more preferably 0.5 or more and 2 or less. The coefficient of thermal expansion of such a reinforcing substrate 90 is preferably 30 ppm/K or more and 80 ppm/K or less. For example, in a case where the conversion layer 14 has CsI:Tl as a material, the coefficient of thermal expansion is 50 ppm/K. In this case, examples of the material relatively close to the conversion layer 14 include PVC, acrylic, PET, PC, Teflon (registered trademark), and the like. Moreover, considering the above-described bending elastic modulus, the material of the reinforcing substrate 90 is more preferably a material containing at least one of PET or PC. Additionally, from the viewpoint of elasticity, the reinforcing substrate 90 preferably contains a material having a yield point.

The reinforcing substrate 90 of the present modification example is a substrate having plastic as a material. In a case where the plastic used as the material for the reinforcing substrate 90 is preferably a thermoplastic resin for the above-described reasons, and include at least one of PC, PET, styrol, acrylic, polyacetase, nylon, polypropylene, ABS, engineering plastics, or polyphenylene ether. In addition, the reinforcing substrate 90 is even more preferably at least one of polypropylene, ABS, engineering plastics, PET, or polyphenylene ether among these, is more preferably at least one of styrol, acrylics, polyacetase, or nylon, and is more preferably at least one of PC or PET.

In addition, in a case where the radiation detector 10 comprises the rigid plate 50 and the reinforcing substrate 90, the specific characteristics and materials of the rigid plate 50 and the reinforcing substrate 90 may be the same or different.

The pressure sensitive adhesive 92 is provided on the entire surface of the reinforcing substrate 90 facing the sensor substrate 12, and the reinforcing substrate 90 is provided on the conversion layer 14, specifically, on the reflective layer 62 that covers the conversion layer 14, by the pressure sensitive adhesive 92.

The step of providing the reinforcing substrate 90 on the conversion layer 14 may be performed after the peeling step (refer to FIG. 5C) but is preferably performed before the peeling step. In a case where the sensor substrate 12 provided with the conversion layer 14 is peeled from the support body 400, the base material 11 is deflected. In a case where the base material 11 is deflected, there is a concern that the conversion layer 14, particularly the end part of the conversion layer 14, may be peeled from the base material 11. In contrast, in a case where the sensor substrate 12 having the reinforcing substrate 90 provided on the conversion layer 14 is peeled from the support body 400, it is possible to suppress the peeling of the conversion layer 14 from the base material 11, which is caused by the deflection of the base material 11 in order to reinforce the bending stiffness of the base material 11.

In addition, in the radiation detector 10 shown in FIG. 12A, an example in which the size (area) of the reinforcing substrate 90 is the same as that of the base material 11, and the positions of the end part of the reinforcing substrate 90 and the end part of the base material 11 are the same is illustrated. However, the size of the reinforcing substrate 90 and the position of the end part are not limited to the present example. For example, as shown in FIG. 12B, a configuration may be adopted in which the reinforcing substrate 90 is larger than the base material 11. In addition, the specific size of the reinforcing substrate 90 can be determined depending on the size of the inside of the housing 120 that houses the radiation detector 10, and the like. Additionally, as shown in FIG. 12B, the end part of the reinforcing substrate 90 is located outside an end part of the base material 11, that is, the sensor substrate 12.

In this way, by making the size of the reinforcing substrate 90 larger than the size of the base material 11, for example, for example, in a case where an impact is applied to the housing 120 and a side surface (a surface intersecting the irradiation surface 120A) of the housing 120 is recessed such that the radiographic imaging apparatus 1 is dropped, the reinforcing substrate 90 interferes with the side surface of the housing 120. On the other hand, since the sensor substrate 12 is smaller than the reinforcing substrate 90, the sensor substrate 12 is less likely to interfere with the side surface of the housing 120. Therefore, according to the radiation detector 10 shown in FIG. 12B, it is possible to suppress the influence of the impact applied to the radiographic imaging apparatus 1 on the sensor substrate 12.

In addition, from the viewpoint of suppressing the influence of the impact of the reinforcing substrate 90 applied to the radiographic imaging apparatus 1 on the sensor substrate 12, as shown in FIG. 12B, at least a part of the end part of the reinforcing substrate 90 may protrude further outward than the end part of the base material 11. For example, even in a case where the size of the reinforcing substrate 90 is smaller than the size of the base material 11, the end part of the reinforcing substrate 90 that protrudes further outward than the end part of the base material 11 interferes with the side surface of the housing 120. Therefore, the influence of the impact on the sensor substrate 12 can be suppressed.

Additionally, for example, as shown in FIGS. 12C and 12D, a configuration may be adopted in which the reinforcing substrate 90 is smaller than the base material 11. In the example shown in FIG. 12C, the reinforcing substrate 90 is not provided at the position facing the terminal 113. That is, the area of the reinforcing substrate 90 in the radiation detector 10 of the present modification example is smaller than a value obtained by subtracting the area of a region where the terminal 113 is provided from the area of the base material 11. On the other hand, in an example shown in FIG. 12D, the end part of the reinforcing substrate 90 is located at the peripheral edge part 14B of the conversion layer 14, and the conversion layer 14 is provided in a region narrower than a region where the reinforcing substrate 90 covers the first surface 11A of the base material 11.

Removing the flexible cable 112 or a component electrically connected to the base material 11 (sensor substrate 12) and newly reconnecting the component due to a defect or a positional deviation is referred to as rework. In this way, by making the size of the reinforcing substrate 90 smaller than the size of the base material 11, the rework can be performed without being disturbed by the end part of the reinforcing substrate 90. Therefore, the rework of the flexible cable 112 can be facilitated.

Additionally, for example, as shown in FIG. 12E, the reinforcing substrate 90 may be provided in a state of being bent along an inclined surface in the peripheral edge part 14B of the conversion layer 14. In the example shown in FIG. 12E, the adhesive layer 64 and the protective layer 66 also cover the portion that covers the first surface 11A of the base material 11 and the first surface 11A of the base material 11 outside the portion. That is, the end parts of the adhesive layer 64 and the protective layer 66 are sealed with the reinforcing substrate 90. The portion of the reinforcing substrate 90 that extends onto the base material 11 is bonded to the base material 11 via the pressure sensitive adhesive 92. By covering the end parts of the adhesive layer 64 and the protective layer 66 with the reinforcing substrate 90 in this way, the peeling of the protective layer 66 can be suppressed.

### Modification Example 3

In the present modification example, a configuration in which the periphery of the conversion layer 14 in the radiation detector 10 is sealed will be described with reference to FIG. 13. FIG. 13 shows an example of a cross-sectional view of a radiation detector 10 of the present modification example, which corresponds to the sectional view taken along the line A-A of the radiation detector 10 shown in FIG. 3A.

As shown in FIG. 13, a configuration may be adopted in which the peripheral edge part 14B of the conversion layer 14 is sealed by a sealing member 70. In the example shown in FIG. 13, the sealing member 70 is provided in a space created by the base material 11, the conversion layer 14, and the reinforcing substrate 90 as described above. Specifically, a sealing member 70 is provided in a space formed between the conversion layer 14 (protective layer 66) and the reinforcing substrate 90 in the region corresponding to the peripheral edge part 14B of the conversion layer 14 and the region further outside thereof. The material of the sealing member 70 is not particularly limited, and for example, resin can be used.

The method of providing the sealing member 70 is not particularly limited. For example, the reinforcing substrate 90 may be provided on the conversion layer 14 covered with a pressure-sensitive adhesive layer 60, the reflective layer 62, the adhesive layer 64, and the protective layer 66 by the pressure sensitive adhesive 92, and then, the sealing member 70 having fluidity may be injected into the space formed between the conversion layer 14 (protective layer 66) and the reinforcing substrate 90 to cure the reinforcing substrate 90. Additionally, for example, after the conversion layer 14, the pressure-sensitive adhesive layer 60, the reflective layer 62, the adhesive layer 64, and the protective layer 66 are sequentially formed on the base material 11, the sealing member 70 may be formed, and the reinforcing substrate 90 may be provided by the pressure sensitive adhesive 92 in a state where the conversion layer 14 and the sealing member 70 covered with the pressure-sensitive adhesive layer 60, the reflective layer 62, the adhesive layer 64, and the protective layer 66.

Additionally, the region where the sealing member 70 is provided is not limited to the configuration shown in FIG. 13. For example, the sealing member 70 may be provided on the entire first surface 11A of the base material 11, and the terminal 113 to which the flexible cable 112 is electrically connected may be sealed together with the flexible cable 112.

In this way, by filling the space formed between the conversion layer 14 and the reinforcing substrate 90 with the sealing member 70 and sealing the conversion layer 14, the peeling of the reinforcing substrate 90 from the conversion layer 14 can be suppressed. Moreover, since the conversion layer 14 has a structure in which the conversion layer 14 is fixed to the sensor substrate 12 by both the reinforcing substrate 90 and the sealing member 70, the stiffness of the base material 11 is further reinforced.

### Modification Example 4

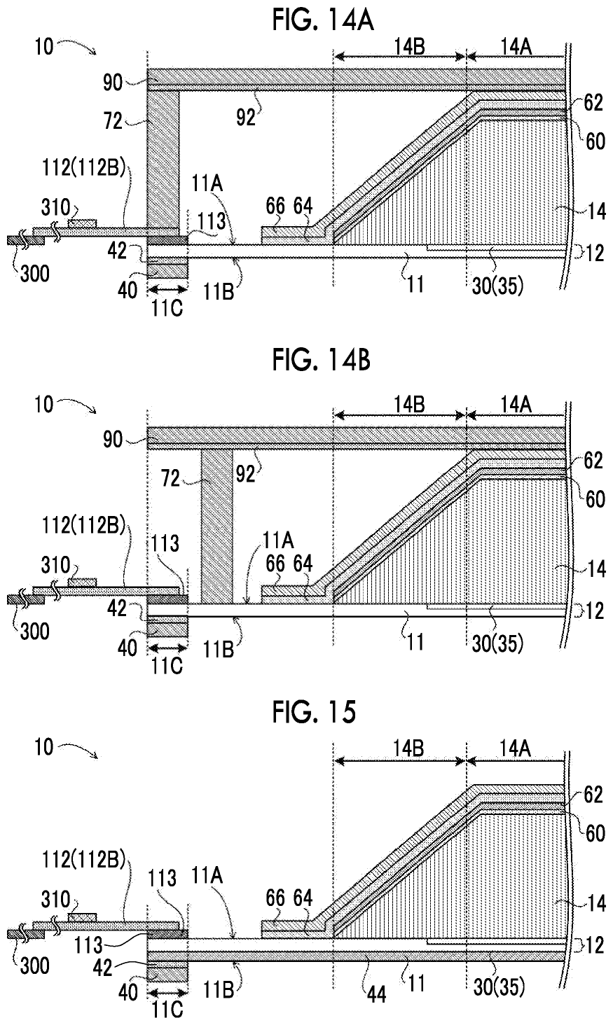

In the present modification example, a configuration in which the reinforcing substrate 90 in the radiation detector 10 is supported by the support member 72 will be described with reference to FIGS. 14A and 14B. Each of FIGS. 14A and 14B shows an example of a cross-sectional view of a radiation detector 10 of the present modification example, which corresponds to the sectional view taken along the line A-A of the radiation detector 10 illustrated in FIG. 3A.

In the radiation detector 10 shown in FIG. 14A, the end part of the reinforcing substrate 90 is supported by the support member 72. That is, one end of the support member 72 is connected to the flexible cable 112 or the first surface 11A of the base material 11, and the other end of the support member 72 is connected to the end part of the reinforcing substrate 90 by the pressure sensitive adhesive 92. In addition, the support member 72 may be provided on the entire outer edge part of the base material 11 or may be provided on a portion of the outer edge. In this way, by supporting the end part of the reinforcing substrate 90 that extends while forming the space between the reinforcing substrate 90 and the base material 11 with the support member 72, the conversion layer 14 can be inhibited from being peeled from the sensor substrate 12. Additionally, by providing the support member 72 on the flexible cable 112 connected to the terminal 113, it is possible to inhibit the flexible cable 112 from being peeled from the terminal 113.

On the other hand, in the radiation detector 10 shown in FIG. 14B, a position inside the end part of the reinforcing substrate 90 is supported by the support member 72. In the example shown in FIG. 14B, the position where the support member 72 is provided is only outside the region where the flexible cable 112 and the terminal 113 are provided. In the example shown in FIG. 14B, one end of the support member 72 is connected to the first surface 11A of the base material 11, and the other end of the support member 72 is connected to the end part of the reinforcing substrate 90 by the pressure sensitive adhesive 92. In this way, by not providing the support member 72 on the flexible cable 112 and the terminal 113, the rework of the flexible cable 112 can be facilitated.

In this way, according to the radiation detector 10 of the present modification example, by supporting the reinforcing substrate 90 with the support member 72, the stiffness reinforcing effect of the reinforcing substrate 90 can be obtained up to the vicinity of the end part of the base material 11, and the effect of suppressing the deflection of the material 11 can be exerted. For that reason, according to the radiation detector 10 of the present modification example, it is possible to suppress the peeling of the conversion layer 14 from the sensor substrate 12.

In addition, in a case where the present modification example and the above Modification Example 3 are combined with each other, in other words, in a case where the radiation detector 10 comprises the sealing member 70 and the support member 72, a part or the whole of the space surrounded by the support member 72, the reinforcing substrate 90, the conversion layer 14, and the base material 11 may be filled with the sealing member 70 and may be sealed by the sealing member 70.

### Modification Example 5

In the present modification example, a configuration in which the radiation detector 10 comprises an antistatic layer 44 will be described with reference to FIG. 15. FIG. 15 shows an example of a cross-sectional view of a radiation detector 10 of the present modification example, which corresponds to the sectional view taken along the line A-A of the radiation detector 10 shown in FIG. 3A.

As shown in FIG. 15, in the radiation detector 10 of the present modification example, the antistatic layer 44 is provided on the second surface 11B of the base material 11. The reinforcing member 40 is provided on the surface of the antistatic layer 44 opposite to a surface on the second surface 11B side by the pressure sensitive adhesive 42. In other words, the reinforcing member 40, the pressure sensitive adhesive 42, the antistatic layer 44, and the base material 11 are laminated in this order.

The material of the antistatic layer 44 has a function of suppressing the influence of electromagnetic wave noise, static electricity, and the like from the outside. As the antistatic layer 44, for example, a laminated film of a resin film such as Alpet (registered trademark) and a metal film, an antistatic paint “Colcoat” (product name: made by Colcoat), PET, polypropylene, and the like can be used.

In addition, a region where the antistatic layer 44 is provided may be a region that covers at least the pixel region 35 and is not limited to the configuration shown in FIG. 15. For example, a configuration may be adopted in which the antistatic layer 44 is provided only in the region where the reinforcing member 40 is provided.

In this way, according to the radiation detector 10 of the present modification example, since the antistatic layer 44 is provided on the second surface 11B of the base material 11, charging of the sensor substrate 12 is suppressed, and the influence of static electricity can be suppressed.

### Modification Example 6

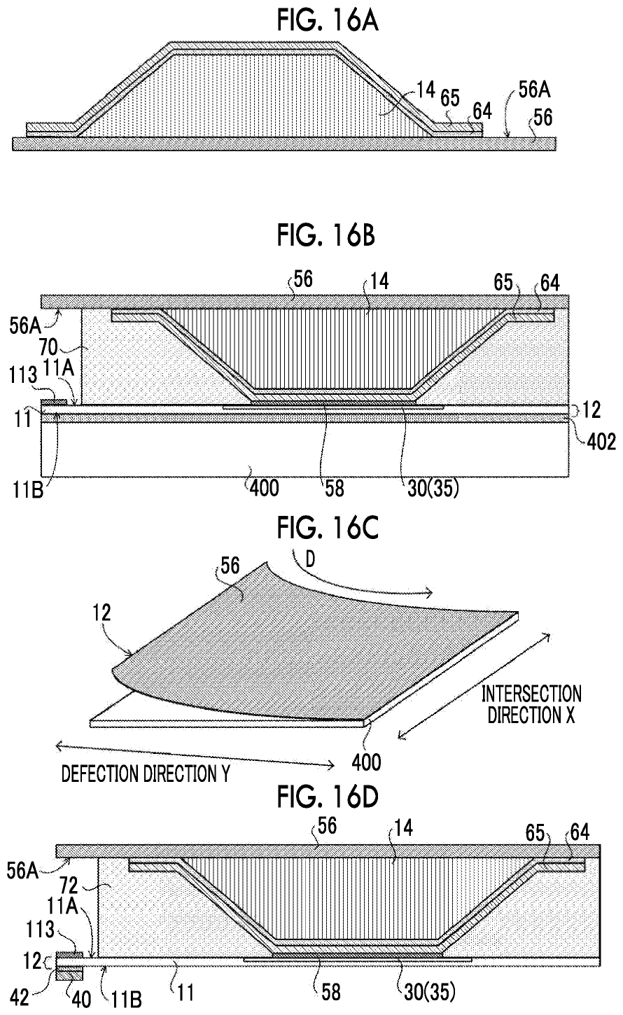

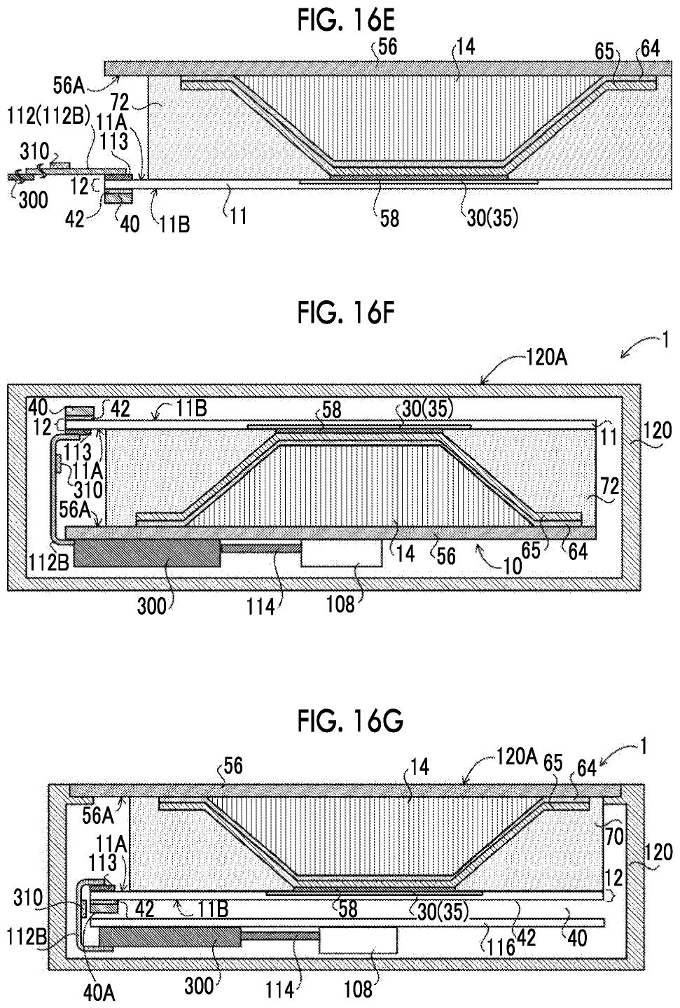

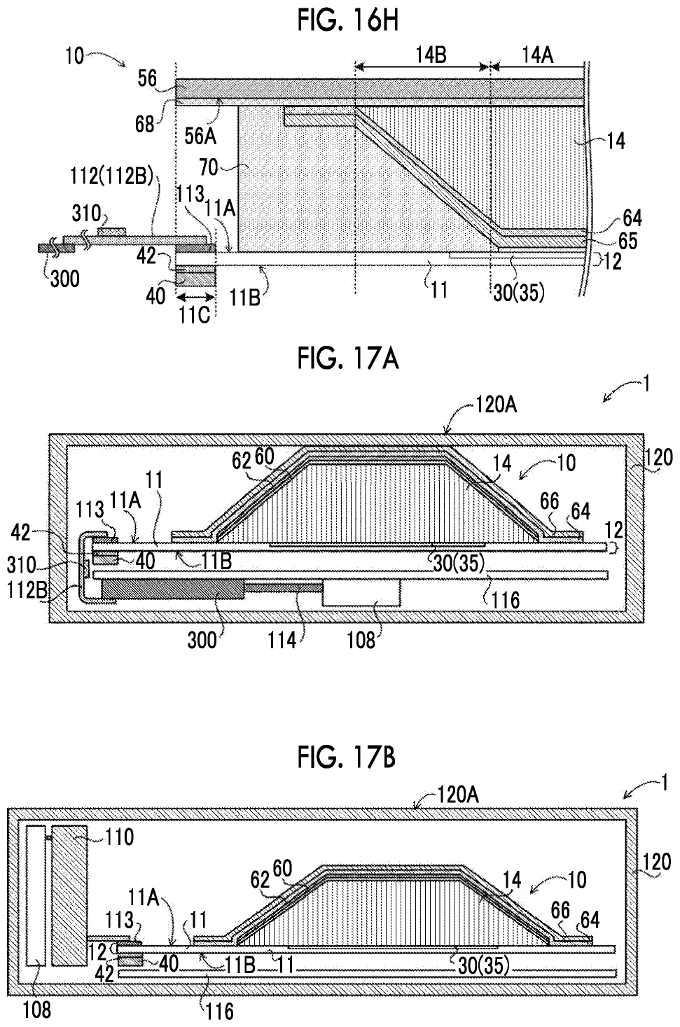

In the present modification example, a modification example of the method of manufacturing the radiographic imaging apparatus 1 will be described with reference to FIGS. 16A to 16H. In addition, the radiographic imaging apparatus 1 of the present modification example includes a method of manufacturing the radiation detector 10.

Since the step of forming the sensor substrate 12 is the same as the step described above with reference to FIG. 5A, the description thereof will be omitted.

Additionally, in the present modification example, as shown in FIG. 16A, the conversion layer 14 is formed on the first surface 56A of the substrate 56. In the present embodiment, the conversion layer 14 of CsI is directly formed as a columnar crystal on the first surface 56A of the substrate 56 by vapor-phase deposition methods, such as a vacuum vapor deposition method, a sputtering method, and a CVD method. In this case, the side of the conversion layer 14, which is in contact with the first surface 56A of the substrate 56, is a growth-direction base point side of the columnar crystal. The substrate 56 is a substrate for forming the conversion layer 14, and is, for example, a vapor deposition substrate. As materials of the substrate 56, for example, a resin such as PET, a metal containing at least one of Mg, Al, Li, carbon, or the like are preferable, and a material containing carbon as a main component is more preferable.

After the conversion layer 14 is formed on the substrate 56, the adhesive layer 64 and the protective layer 66 are provided so as to cover the conversion layer 14. In addition, in the present configuration, as shown in FIG. 16A and the like, unlike each of the above radiation detectors 10, the pressure-sensitive adhesive layer 60 and the reflective layer 62 are not provided on the conversion layer 14. In addition, it is preferable to cover all the substrate 56, the conversion layer 14, the adhesive layer 64, and the protective layer 66 with a moisture proof film (not shown).

In addition, any step may be performed first, regardless of the order of the step of forming the sensor substrate 12 described with reference to FIG. 5A and the step of forming the conversion layer 14 described with reference to FIG. 16A, or both the steps may be performed in parallel.

Next, as shown in FIG. 16B, the conversion layer 14 is provided on the first surface 11A of the base material 11. In the present embodiment, by the pressure-sensitive adhesive layer 58, the conversion layer 14 is provided on the first surface 11A of the base material 11 by the pressure-sensitive adhesive layer 58 in a state where the upper side of the conversion layer 14, more specifically, the side opposite to the side of the conversion layer 14 in contact with the substrate 56 faces the first surface 11A of the base material 11.

Additionally, a space between the substrate 56 and the sensor substrate 12 is sealed by the sealing member 70. The method of sealing between the substrate 56 and the sensor substrate 12 with the sealing member 70 is not particularly limited. For example, after the conversion layer 14 is provided on the sensor substrate 12, the sealing member 70 having fluidity may be injected into the space formed between the sensor substrate 12 and the conversion layer 14 (protective layer 66) to cure the sealing member 70.

In addition, the method of providing the conversion layer 14 on the sensor substrate 12 is not limited to the method of performing bonding by the pressure-sensitive adhesive layer 58.

An uncured sealing member 70 is provided in a region extending from the peripheral edge part 14B of the conversion layer 14 formed on the substrate 56 to the first surface 56A of the substrate 56, and the support member 72 described in the above Modification Example 4 is provided, and the conversion layer 14 in this state is disposed on the first surface 11A of the base material 11.

In this state, an internal space formed by the base material 11, the substrate 56, the sealing member 70, and the support member 72 is pressure-reduced to, for example, a pressure, such as 0.2 atm to 0.5 atm, which is lower than the atmospheric pressure, using a pressure-reducing pump or the like. In this way, by making the internal space formed by the base material 11, the substrate 56, the sealing member 70, and the support member 72 lower than the atmospheric pressure, the base material 11 (sensor substrate 12) and the substrate 56 are pressed from the outside to the internal space side at the atmospheric pressure. The conversion layer 14 is provided on the first surface 11A of the base material 11 by pressing the base material 11 and the substrate 56 at the atmospheric pressure. Therefore, the conversion layer 14 and the base material 11 closely adhere to each other without providing the pressure-sensitive adhesive layer 58.

After that, as shown in FIG. 16C, the sensor substrate 12 provided with the conversion layer 14 is peeled from the support body 400. This peeling step can be the same as the above-described peeling step described with reference to FIG. 5C. In addition, in a case where the substrate 56 having carbon as a main component is used, since the substrate 56 is difficult to be deflected, laser peeling may be performed instead of mechanical peeling. Additionally, in the laser peeling, the sensor substrate 12 is peeled from the support body 400 by radiating a laser beam from a back surface (a surface opposite to the surface on which the sensor substrate 12 is provided) of the support body 400 and by decomposing the peeling layer 402 with the laser beam transmitted through the support body 400.

Next, as shown in FIG. 16D, the reinforcing member 40 provided with the pressure sensitive adhesive 42 is bonded to the facing region 11C of the second surface 11B of the base material 11.

Next, as shown in FIG. 16E, the flexible cable 112 is electrically connected to the sensor substrate 12. Specifically, the flexible cable 112 on which the driving IC 210 or the signal processing IC 310 is mounted is thermocompression-bonded to the terminal 113 to electrically connect the terminal 113 and the flexible cable 112. Accordingly, the flexible cable 112 is electrically connected to the sensor substrate 12.

Moreover, as shown in FIG. 16F or FIG. 16G by housing the radiation detector 10, the circuit unit, and the like in the housing 120, the radiographic imaging apparatus 1 is manufactured. The radiographic imaging apparatus 1 shown in FIG. 16F shows a cross-sectional view of an example of an ISS type radiographic imaging apparatus 1. Additionally, the radiographic imaging apparatus 1 shown in FIG. 16G shows a cross-sectional view of an example of the PSS type radiographic imaging apparatus 1. Additionally, in the radiographic imaging apparatus 1 shown in FIG. 16G an example of a configuration in which the substrate 56 is adopted as a top plate on the irradiation surface 120A side of the housing 120 is shown. In this case, as shown in FIG. 16G the size of the substrate 56 is larger than that of the sensor substrate 12, and an end part of the substrate 56 protrudes outward from the end part of the sensor substrate 12. In the radiographic imaging apparatus 1 shown in FIG. 16Q the radiation detector 10 is housed inside the housing 120 by fitting the substrate 56 into an opening portion of the housing 120 that has an opening shape on a top plate portion on the irradiation surface 120A side. In this way, by using the substrate 56 of the conversion layer 14 as the top plate of the housing 120, the thickness of the housing 120, more specifically, the thickness in a radiation transmission direction can be further reduced, and the radiographic imaging apparatus 1 can be slimmed. Additionally, since the top plate of the housing 120 itself is unnecessary, the weight of the radiographic imaging apparatus 1 can be further reduced.

In this way, according to the present modification example, the radiation detector 10 can be manufactured without directly vapor-deposing the conversion layer 14 on the sensor substrate 12.

In addition, in the case of the manufacturing method of the present modification example, it is preferable to provide the reflective layer 68 between the substrate 56 and the conversion layer 14, as shown in FIG. 16H. In an example shown in FIG. 16H, the reflective layer 68 covers the entire first surface 56A of the substrate 56. The reflective layer 68 has a function of reflecting the light converted by the conversion layer 14, similarly to the above-described reflection layer 62. For that reason, the same material as the above-described reflective layer 62 can be applied to the reflective layer 68 of the present modification example.

### Modification Example 7

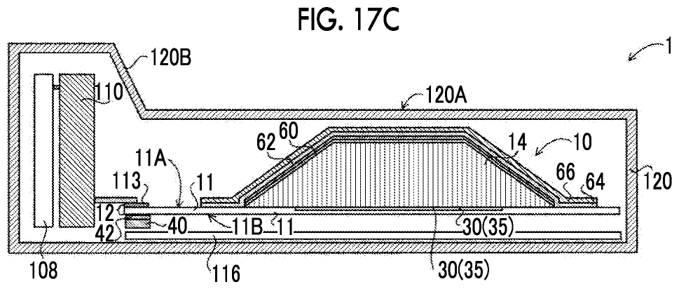

In the present modification example, a modification example, in a housed state, of the radiation detector 10 in the radiographic imaging apparatus 1 will be described with reference to FIGS. 17A to 17C. Each of FIGS. 17A to 17C is an example of cross-sectional views of a radiographic imaging apparatus 1 of the present modification example.

FIG. 17A shows an example of a configuration in which the radiation detector 10 is in contact with an inner wall surface of the top plate on the irradiation surface 120A side of the housing 120. In the example shown in FIG. 17A, the conversion layer 14 is in contact with the inner wall surface of the top plate on the irradiation surface 120A side of the housing 120. In addition, in a case where the radiation detector 10 comprises the reinforcing substrate 90 as in the above-described Modification Example 1 and the like, a configuration is obtained in which the reinforcing substrate 90 is in contact with the inner wall surface of the top plate on the irradiation surface 120A side of the housing 120.

In this case, the radiation detector 10 and the inner wall surface of the housing 120 may be bonded to each other via an adhesive layer, or may simply be in contact with each other without an adhesive layer. Since the radiation detector 10 and the inner wall surface of the housing 120 are in contact with each other in this way, the stiffness of the radiation detector 10 is further secured.

Additionally, a configuration in which circuit units such as the radiation detector 10, the control substrate 110, and the power source unit 108 are disposed in the transverse direction in the drawing is exemplified in FIG. 17B. In other words, in the radiographic imaging apparatus 1 shown in FIG. 17B, the radiation detector 10 and the circuit unit are disposed side by side in a direction intersecting the irradiation direction of the radiation.

In addition, although FIG. 17B shows a configuration in which both the power source unit 108 and the control substrate 110 are provided on one side of the radiation detector 10, specifically, on one side of a rectangular pixel region 35, a position where the circuit units such as the power source unit 108 and the control substrate 110 are provided is not limited to the configuration shown in FIG. 17B. For example, the circuit units such as the power source unit 108 and the control substrate 110 may be provided so as to be respectively distributed onto two facing sides of the pixel region 35 or may be provided so as to be respectively distributed onto two adjacent sides. In this way, by disposing the radiation detector 10 and the circuit unit side by side in the direction intersecting the irradiation direction of the radiation, the thickness of the housing 120, more specifically, the thickness in the direction in which the radiation is transmitted can be further reduced, and the radiographic imaging apparatus 1 can be slimmed.

Additionally, in a case where the radiation detector 10 and the circuit unit are disposed side by side in a direction intersecting the radiation irradiation direction, the thickness of the housing 120 may be different between the portion of the housing 120 in which each of the circuit units such as the power source unit 108 and a control substrate 110 are provided and the portion of the housing 120 in which the radiation detector 10 is provided, as in the radiographic imaging apparatus 1 shown in FIG. 17C.

As shown in the example shown in FIGS. 17B and 17C, there are many cases where the circuit units of the power source unit 108 and the control substrate 110 are thicker than the radiation detector 10. In such a case, as in the example shown in FIG. 17C, the thickness of the portion of the housing 120 in which the radiation detector 10 is provided may be smaller than the thickness of the portion of the housing 120 in which each of the circuit units such as the power source unit 108 and the control substrate 110 is provided. According to the radiographic imaging apparatus 1 shown in FIG. 17C, it is possible to configure an ultra-thin radiographic imaging apparatus 1 according to the thickness of the radiation detector 10.

In addition, as in the example shown in FIG. 17C, in a case where the thickness of the portion of the housing 120 in which each of the circuit units such as the power source unit 108 and the control substrate 110 is provided and the thickness of the portion of the housing 120 in which the radiation detector 10 is provided are made different, and in a case where a step is generated at a boundary portion between the two portions, there is a concern that a sense of discomfort may be given to a subject who comes into contact with a boundary portion 120B. For that reason, it is preferable that the form of the boundary portion 120B has an inclination. Additionally, the portion of the housing 120 in which each of the circuit units such as the power source unit 108 and the control substrate 110 is housed and the portion of the housing 120 in which the radiation detector 10 is housed may be formed of different materials.

As described above, each of the above radiation detectors 10 comprises the sensor substrate 12, the conversion layer 14, and the reinforcing member 40. In the sensor substrate 12, the plurality of pixels 30 that accumulate electric charges generated in response to the light converted from the radiation are formed in the pixel region 35 of the first surface 11A of the flexible base material 11, and the first surface 11A is provided with the terminal 113 for electrically connecting the flexible cable 112. The conversion layer 14 is provided on the first surface 11A of the base material 11 and converts radiation into light. The reinforcing member 40 is provided in a region including at least the facing region 11C, facing the terminal 113, on the second surface 11B of the base material 11 opposite to the first surface 11A and has super engineering plastic as a material. Alternatively, the reinforcing member 40 is provided in a region including at least the facing region 11C, facing the terminal 113, on the second surface 11B of the base material 11 opposite to the first surface 11A and has a resin with a continuous operating temperature of 150° C. or higher as a main material.

In each of the above radiation detectors 10, the reinforcing member 40 is provided in the region including at least the facing region 11C of the second surface 11B of the base material 11. Therefore, in a case where the flexible cable 112 is electrically connected to the terminal 113, including the case of rework, the bending stiffness of the base material 11 in the vicinity of the terminal 113 is reinforced by the reinforcing member 40. For that reason, in each of the above-described radiation detectors 10, the handleability is improved.

Additionally, heat is applied to the base material 11 by the heat treatment performed in a case where the flexible cable 112 is electrically connected to the terminal 113, including the case of rework. The heat applied to the base material 11 due to this heat treatment mainly tends to propagate from the facing region 11C of the second surface 11B to the reinforcing member 40. In a case where the heat propagates to the reinforcing member 40, there is a case where the reinforcing member 40 is deformed by the propagated heat.

However, in each of the above radiation detectors 10, the reinforcing member 40 having high heat resistance is provided in the region including at least the facing region 11C of the second surface 11B of the base material 11. For that reason, in each of the above-described radiation detectors 10, the deformation of the reinforcing member 40 caused by the heat propagated from the base material 11 can be suppressed.

Therefore, each of the above-described radiation detectors 10 has excellent handleability, and the deformation of the reinforcing member caused by the heat applied to a terminal can be suppressed.

In addition, the configurations of the radiographic imaging apparatus 1 and the radiation detector 10, and the method of manufacturing the radiation detector 10 are not limited to the configurations described with reference to FIGS. 1 to 17C. For example, as shown in FIG. 1, an aspect in which the pixels 30 are two-dimensionally arranged in a matrix has been described. However, the invention is not limited, and the pixels 30 may be one-dimensionally arranged or may be arranged in a honeycomb arrangement. Additionally, the shape of the pixels is also not limited, and may be a rectangular shape, or may be a polygonal shape, such as a hexagonal shape. Moreover, the shape of the pixel region 35 is also not limited.

In addition, the configurations, manufacturing methods, and the like of the radiographic imaging apparatuses 1, the radiation detectors 10, and the like in the above embodiments and respective modification examples are merely examples, and can be modified in accordance with situations without departing from the scope of the present invention.

The disclosure of Japanese Patent Application No. 2020-027529 filed on Feb. 20, 2020 is incorporated in the present specification by reference in its entirety.

All documents, patent applications, and technical standards described in the present specification are incorporated in the present specification by reference in their entireties to the same extent as in a case where the individual documents, patent applications, and technical standards are specifically and individually written to be incorporated by reference.

What is claimed is:

1. A radiation detector comprising:
a substrate in which a plurality of pixels that accumulate electric charges generated in response to light converted from radiation are formed in a pixel region of a first surface of a flexible base material and the first surface is provided with a terminal for electrically connecting a cable;
a conversion layer that is provided on a first surface side of the base material and converts the radiation into the light; and
a reinforcing member that is provided in a region including at least a facing region, facing the terminal, on a second surface of the base material opposite to the first surface and includes a resin in which a continuous operating temperature based on UL 746B regulations is 150° C. or higher.

2. A radiation detector comprising:
a substrate in which a plurality of pixels that accumulate electric charges generated in response to light converted from radiation are formed in a pixel region of a first surface of a flexible base material and the first surface is provided with a terminal for electrically connecting a cable;
a conversion layer that is provided on a first surface side of the base material and converts the radiation into the light; and
a reinforcing member that is provided in a region including at least a facing region, facing the terminal, on a second surface of the base material opposite to the first surface and has super engineering plastic as a material.

3. The radiation detector according to claim 1,
wherein the reinforcing member has at least one of a resin having a sulfonyl group, a resin having a phenylene sulfide structure, a resin having an imide group, a resin having an arylene ether structure and an arylene ketone structure, or a resin having a benzimidazole structure as a main material.

4. The radiation detector according to claim 1,
wherein the reinforcing member includes at least one of polysulfone, polyethersulfone, polyphenylene sulfide, polyetheretherketone, or tetrafluoroethylene-ethylene copolymer as a material.

5. The radiation detector according to claim 1,
wherein the reinforcing member includes at least one of polysulfone, polyethersulfone, polyphenylene sulfide, polyamidoimide, polyetheretherketone, polyimide, polybenzoimidazole, thermoplastic polyimide, or tetrafluoroethylene-ethylene copolymer as a material.

6. The radiation detector according to claim 1,
wherein the reinforcing member includes at least one of polysulfone, polyethersulfone, polyphenylene sulfide, polyamidoimide, polyetheretherketone, polyimide, polybenzoimidazole, thermoplastic polyimide, tetrafluoroethylene-ethylene copolymer, polyphenyl sulfone, polyarylate, polyetherimide, liquid crystal polymer, polytetrafluoroethylene, tetrafluoroethylene-hexafluoropropylene copolymer, tetrafluoroethylene-perfluoroalkylvinylether copolymer, polychlorotrifluoroethylene, or polyvinylidene fluoride as a material.

7. The radiation detector according to claim 1,
wherein a bending stiffness of the reinforcing member is higher than that of the base material.

8. The radiation detector according to claim 1,
wherein the reinforcing member is provided in a region of the second surface including the facing region and a part of a region facing a region where the conversion layer is provided.

9. The radiation detector according to claim 1, further comprising:
a reinforcing member that is provided in a region where the reinforcing member is not provided, on the second surface of the base material, and has a higher bending stiffness than that of the base material.

10. A radiographic imaging apparatus comprising:
the radiation detector according to claim 1; and
a circuit unit for reading out electric charges accumulated in the plurality of pixels.

11. A method of manufacturing a radiation detector, the method comprising:
forming a substrate in which a flexible base material is provided on a support body, a plurality of pixels that accumulate electric charges generated in response to light converted from radiation are formed in a pixel region of a first surface of the base material, and the first surface is provided with a terminal for electrically connecting a cable;
providing a conversion layer that converts the radiation into the light, on the first surface of the base material;
peeling the substrate provided with the conversion layer from the support body; and
providing a reinforcing member having super engineering plastic as a material in a region including at least a facing region, facing the terminal, on a second surface of the base material opposite to the first surface.

12. A method of manufacturing a radiation detector, the method comprising:
forming a substrate in which a flexible base material is provided on a support body, a plurality of pixels that accumulate electric charges generated in response to light converted from radiation are formed in a pixel region of a first surface of the base material, and the first surface is provided with a terminal for electrically connecting a cable;
providing a conversion layer that converts the radiation into the light, on the first surface of the base material;
peeling the substrate provided with the conversion layer from the support body; and
providing a reinforcing member having a resin in which a continuous operating temperature based on UL 746B regulations is 150° C. or higher in a region including at least a facing region, facing the terminal, on the second surface of the base material opposite to the first surface.

13. The method of manufacturing a radiation detector according to claim 11, further comprising:
electrically connecting the cable to the terminal after the reinforcing member is provided.
